(19) United States
(12) Patent Application Publication
(10) Pub. No.: US 2012/0084887 A1
(43) Pub. Date: Apr. 5, 2012

(54) METHOD AND COMPOSITION FOR INCREASING PLANT SURVIVAL & VIABILITY UNDER COLD STORAGE, OR DARK AND COLD STORAGE CONDITIONS

(76) Inventors: Richard James McAvoy, Mansfield Center, CT (US); Yi Li, Mansfield, CT (US); Mariya Khodakovskaya, Little Rock, AR (US)

(21) Appl. No.: 13/276,470

(22) Filed: Oct. 19, 2011

Related U.S. Application Data
(62) Division of application No. 11/891,026, filed on Aug. 9, 2007, now abandoned.
(60) Provisional application No. 60/837,585, filed on Aug. 15, 2006.

(51) Int. Cl. A01H 5/00; A01H 5/10

(52) U.S. Cl. 800/298

(57) ABSTRACT

Unique fusion genes are disclosed which are useful for transforming a wide range of plants, and when used in tandem, result in a significant alteration of the plant phenotype with respect to tolerance to the stress from prolonged storage under either dark or cold conditions, or a combination of cold and dark conditions. The gene constructs include (1) a cold-regulated gene (COR15a) promoter to drive an ipt coding sequence that expresses IPT in the tissues of plants or plant parts exposed to a short cold induction period; and (2) a cold-regulated gene (COR15a) promoter to drive a (FAD7) coding sequence that expresses a fatty acid desaturase enzyme in the tissues of plants or plant parts exposed to a short cold induction period. Transgenic plants comprising these genes are also disclosed.

### CROSS-REFERENCE TO RELATED APPLICATIONS

This is a divisional application of copending U.S. Non-Provisional application Ser. No. 11/891,026, filed on Aug. 9, 2007, which application claims the benefit of U.S. Provisional Application No. 60/837,583, filed on Aug. 15, 2006. The contents of both aforementioned applications are hereby incorporated by reference.

### REFERENCE TO SEQUENCE LISTING, A TABLE, OR A COMPUTER PROGRAM LISTING COMPACT DISC APPENDIX

Applicants hereby makes reference to the sequence listing file titled MCA-2_Sequence Listing_McAvoy et al_ST25, which is submitted with this application. The sequence listing file consists of the following sequences: SEQ ID NO 1, SEQ ID NO 2, SEQ ID NO 3, SEQ ID NO 4, SEQ ID NO 5, SEQ ID NO 6, SEQ ID NO 7, SEQ ID NO 8, SEQ ID NO 9, SEQ ID NO 10 and SEQ ID NO 11.

### BACKGROUND OF THE INVENTION

Transgenic or recombinant plants are of increasing interest because of the potential to control phenotypic traits as well as to produce large quantities of commercially useful products. Plants have been employed to overproduce heterologous proteins and in principle can produce a wide range of products, including high value proteins and certain pharmaceuticals. Transgenic plants with enhanced production traits such as the ability to survive and thrive after exposure to prolonged environmental stress are particularly desirable as a source of economic benefit to the commercial agricultural plant community including horticulturists, food producers, and agronomist.

Genetically modified plants for agricultural products are already on the market, including herbicide, insect and virus resistant crop plants. Some of the better-known crops engineered for herbicide resistance include soybeans, maize, rapeseed, sugar beet, rice and cotton. Maize, potatoes, tomatoes and cotton have been modified for insect resistance.

Food source plants can be engineered to improve traits that affect nutritional value; for example, elevated iron in rice and wheat, higher amino acid content in potatoes, seedless fruits and increased carotenoids in rice and tomatoes. Recent efforts have turned to produce recombinant plants with maximal desired plant product at a selected harvest time or to significantly increase a desired expressed product by targeting protein product accumulation in a targeted tissues. Of particular interest have been plants engineered to increase oil production; for example, canola oil, which is considered more healthful than trans fats and oils.

Cytokinins regulate a number of growth and developmental processes in plants such as stimulating cell division, maintaining plant vigor, delaying plant senescence and post-harvest and low temperature induced leaf yellowing (Gan and Amasino, 1997). In practice, exogenous cytokinin applications are often not efficacious in commercial horticulture because they are expensive to use and are not readily assimilated (Hobbie et al., 1994).

Li et al. (1992) generated cytokinin overproducing tobacco plants using the ipt gene cloned from Agrobacterium tumefaciens. The ipt gene encodes the enzyme isopentenyl transferase, which catalyzes the rate-limiting step in cytokinin biosynthesis (Akiyoshi et al., 1984). Excised leaves from cytokinin overproducing ipt-transgenic plants displayed prolonged chlorophyll retention and delayed senescence (Li et al., 1992). The delayed senescence observed in intact and excised leaves from ipt transformed plants demonstrates the potential utility of the ipt gene in extending the storage life of ornamental plants, green vegetables, and fruits.

Unfortunately, most ipt transgenic plants exhibit morphological abnormalities, restricting the potential for use in commercial production. This occurs because overproduction of cytokinins during crop growth interferes with normal development (Gan and Amasino, 1997). For example, elevated cytokinins resulted in a reduction in stature, the release from apical dominance, changes in vascular development, and, in many cases, an inhibition of root growth (Klee et al., 1987; Hobbie et al., 1994). To achieve better control of cytokinin overproduction, researchers fused the ipt gene to inducible promoters that were activated by stimuli such as heat (Medford et al., 1989; Smigocki, 1991; Ainley et al., 1993), wounding (Smigocki et al., 1993), or light (Thomas et al., 1995). However in most cases, morphological changes in ipt-transgenic plants were observed even in the absence of an induction stimulus, suggesting leakage of the inducible system. Using the senescence-specific SAG₁₂ promoter to drive ipt expression in tobacco, Gan and Amasino (1995) demonstrated that a specific developmental response could be elicited through more precise control of ipt expression. The SAG₁₂ promoter activated ipt expression only at the onset of senescence, resulting in increased cytokinin concentrations in the senescing tissue and inhibition of the senescence process. Inhibition of leaf senescence by ipt expression led to the attenuation of the senescence-specific promoter, thus preventing cytokinin overproduction that would interfere with other aspects of development. In SAG₁₂-ipt tobacco, leaf senescence was effectively controlled without other developmental abnormalities (Gan and Amasino, 1997). Subsequently, this strategy was successfully used in a variety of plant species including petunia (Clark et al., 2004) and lettuce (McCabe et al., 2001). For example, leaf senescence was retarded in mature 60-d-old lettuce plants that exhibited normal morphology with no significant differences in head diameter or fresh weight of leaves and roots (McCabe et al., 2001).

In commercial horticulture, it is advantageous to be able to store whole live plants (seedlings) and excised shoots (cuttings) for extended periods of time without a loss of vitality. Plants and excised plant parts are typically stored under cool, dark conditions but the incidence of chilling injury and mortality increases with storage duration (Heins et. al., 1995). At warmer temperatures, leaves senesce and overall plant quality deteriorate rapidly in dark storage. Controlled expression of ipt during dark storage, but not during normal crop production, could potentially increase and extend the storage tolerance of commercial crops without adversely affecting subsequent production in the field or glasshouse.

In the past several decades, extensive attention has been paid to the molecular mechanisms of chilling sensitivity in plants because of the agricultural demands for improvements in chilling tolerance of horticultural crops (Graham and Patterson 1982; Nishida and Murata 1996; Iba 2002; Sakamoto and Murata 2002). When comparing the fatty acids in the cellular membranes of chilling resistant versus chilling-sensitive plants, it was found that the chilling resistant plants have a greater abundance of unsaturated fatty acids. During acclimation to cold temperature, the activity of desaturase enzymes increases and the proportion of unsaturated fatty acids rise (Williams et al. 1992; Palta et al. 1993). This modification allows membranes to remain fluid by lowering the temperature at which the membrane lipids experience a gradual phase change from fluid to semi-crystalline. Thus, desaturation of fatty acids provides protection against damage from chilling temperatures.

Several investigators have shown that it is possible to improve the cold resistance of plants by genetic modification. Saturated fatty acid level was greatly decreased in transgenic tobacco plants expressing a Δ9 cyanobacterial desaturase resulting in a significant increase in chilling resistance (Ishizaki-Nishizawa et al. 1996). The cyanobacterial desaturase introduces a cis-double bond at the Δ9-position of both 16- and 18-carbon saturated fatty acids that are linked to various membrane lipids.

In Arabidopsis, three gene products, FAD3, FAD7 and FAD8, mediate the synthesis of trienoic fatty acids from 18:2 and 16:2. Browse and Somerville (1991) reported that a mutation in the FAD7 gene resulted in a temperature-dependent reduction in the 18:3 and 16:3 content in thylakoid-specific lipids. Kodama et al. (1994; 1995) was able to induce a decrease in the dienoic fatty acids (16:2+18:2) and an increase in the trienoic fatty acids (16:3+18:3) in tobacco by overexpressing the Arabidopsis FAD7 gene under the control of the 35S promoter. Evaluation of transgenic and wild type plants showed differences in low-temperature tolerance in young seedlings but no discernible difference in the performance of mature plants. In young seedlings exposed to 1° C. for 7 days and then cultured at 25° C., leaf growth recovered faster in transgenic plants with the FAD7 gene than in wild-type plants. Low-temperature-induced chlorosis was also reduced in the plants transformed with this gene (Kodama et al. 1994). Conversely, tobacco plants with the FAD7 gene silenced had a lower trienoic fatty acid content than the wild type, and were better able to acclimate to higher temperatures (Murakami et al. 2000).

While increased cold tolerance resulted from expression of the 35S-FAD7, transgenic plants with a constitutively expressed desaturase enzyme were not particularly well suited for growth under warm temperatures.

An inducible cold-tolerance trait could have broad application in commercial agriculture, especially in the nursery industry. For instance by inducing the trait, young seedlings (transplants) or vegetative shoot tips (asexual propagules), could be stored at cold temperatures for a prolonged period without high mortality or loss of vigor when returned to normal growth conditions. Recently, Khodakovskaya et al. (2005) reported an increased tolerance to prolonged dark storage in transgenic petunia and chrysanthemum in which a cold-inducible promoter (cor15a) was used to up-regulate cytokinin production via IPT (isopentenyl transferase) gene expression.

In that study, leaves and vegetative shoot tips from IPT plants expressed elevated cytokinin concentrations and retained high chlorophyll concentrations in prolonged dark storage if first exposed to cold-induction conditions. However, if the expression was not induced with a cold signal, the transgenic plants were no more tolerant to dark storage than the wild type plants. In this patent application, we report plant response to FAD7 expression when regulated with a cold temperature signal by using the cold-inducible promoter from the cor15a gene (Baker et al. 1994). Transgenic and wild type plants were subjected to various low temperature stress conditions, and plant survival, chlorophyll concentration, changes in leaf fatty acid composition and chloroplast membrane organization were determined.

In this patent application, we describe a strategy for enhancing plant stress tolerance, survival and subsequent production viability after exposure to prolonged storage under cold and/or dark conditions. This methodology uses double transgenic plants that carry and express both the cor15a-FAD7 and cor15a-IPT transgenes as described herein.

Further, the application demonstrates that plants carrying just the cor15a-FAD7 gene express enhanced tolerance to cold. This single gene trait has many potential benefits for agricultural producers.

### DEFICIENCIES IN THE ART

In commercial horticulture, it is advantageous to be able to store whole live plants (seedlings) and excised shoots (cuttings) for extended periods of time without a loss of vitality. Plants and excised plant parts are typically stored under cool, dark conditions but the incidence of chilling injury and mortality increases with storage duration (Heins et. al., 1995). At wanner temperatures, leaves senesce and overall plant quality deteriorate rapidly in dark storage. Controlled expression of ipt during dark storage, but not during normal crop production, could potentially increase and extend the storage tolerance of commercial crops without adversely affecting subsequent production in the field or glasshouse.

Similarly, plants and excised plant parts that are stored under cold temperatures in addition to the dark conditions, experience a higher incidence of chilling injury and increased mortality with increased storage duration (Heins et al. 1995). Therefore it is important to improve both cold and dark storage tolerance in sensitive species. In the research leading to our invention, we established transgenic Nicotiana alata plants that expressed two independent transgenes, IPT (isopentenyl transferase) and FAD (fatty acid desaturase) that were both controlled by the cold-inducible cor15a promoter from a cold-regulated gene.

Chilling tolerance in plants represents an important agronomic trait. Young transplants or vegetative cuttings that can survive prolonged cold and/or dark storage and then resume vigorous growth allow for greater productivity and greater flexibility for the commercial propagator. A molecular strategy whereby transgenic plants selectively express greater thylakoid membrane stability under cold stress alone or under cold-dark conditions combined, represents a novel approach to selective storage-tolerance in plants.

On the basis of this scenario, we constructed the cor15a-IPT and cor15a-FAD7 fusion genes. We tested the effects of the cor15a-FAD7 gene on membrane stability and long-term survival under cold conditions. Our data demonstrate that cold-induced expression of the trait gene for increased desaturation of chloroplastic membrane fatty acids effectively and dramatically increased seedling survival under prolonged cold storage and that greater thylakoid membrane stability was associated with survival.

In addition, cold-induced plants carrying the cor15a-IPT construct retained high chlorophyll concentrations, coincident with increased cytokinin concentrations, after prolonged exposure to dark conditions (Khodakovskaya et al., 2005).

In the research presented herein, N. alata plants carrying both the cor15a-IPT gene for dark tolerance and the cor15a-FAD7 gene for cold tolerance, were tested under continuous cold, dark storage. As expected the double transgenic plants showed superior tolerance to prolonged dark, cold storage and the trienoic fatty acid (16:3+18:3) components were higher in the cold-tolerance double transgenic lines and lower in the cold-sensitive wild-type line.

How plants perceive temperature in order to regulate membrane fatty acid desaturation remains an open question (Sakamoto and Murata 2002). However, regulation of fatty acid composition via a molecular genetic approach can be immediately beneficial in commercial agriculture.

Our experiments demonstrate that deasaturase activity in transgenic plants can be regulated by a cold signal by using the cold inducible cor15a promoter to drive FAD7 expression. The proportion of trienoic fatty acids in leaves of cor15a-FAD7 plants was higher than in wild-type leaves after long-term exposure to cold, and the fatty profile was correlated with a more stable thylakoid ultrastructure and increased survival under prolonged exposure to cold. Further, combining multiple trait genes (such as the isopentenyl transferase (IPT) gene for dark-tolerance and the FAD7 gene for cold-tolerance, under the control of the same cold-inducible promoter can be used to confer selective tolerance to multiple stress conditions.

### BRIEF SUMMARY OF THE INVENTION

The present invention provides methods that address production and post-harvest deficiencies associated with many types of commercial crops, both horticultural and agronomic, by providing transgenic plants with highly desirable phenotypes. The methods take advantage of the normal endogenous controls for development in the wild-type plant. It has been shown that a plant transformed with the described expression vector is capable of altering both the normal cytokinin content in the plant and the composition of fatty acids in the cellular membranes (especially the thylakoidal membranes of the chloroplasts) when the plant is exposed to a specific environmental stimulus. The simultaneous expression of these transgenes results in predictable phenomic response, typically observed as prolonged chlorophyll retention under dark conditions, and increase survival and recovery after prolonged storage under cold conditions.

In our study, the cor15a gene promoter was selected to drive FAD7 expression so that an increase in isopentenyl transferase (leading to increased cytokinin) and/or increased fatty acid desaturation, would occur after the plants were exposed to a brief but specific environmental signal. The cor15a gene is a member of the COR (cold-regulated) gene family. Arabidopsis cor15a gene is cold regulated, has CRT/DRE regulatory elements, and is induced in response to the CBF transcriptional activators (Thomashow 1999; Thomashow 2001; Jaglo et al. 2001). Cor15a is inactive, or very weakly active, in most plant tissues and plant organs maintained under normal grown temperatures but becomes highly active in plant shoots in response to low temperature (Baker et al. 1994).

Previously, Khodakovskaya et al. (2005) used the cor15a promoter to drive expression of the IPT gene (resulting in increased tissue cytokinin concentrations) in petunia and chrysanthemum, and demonstrated that IPT expression could be regulated in response to a short (3-d) cold treatment. In that study, IPT expression did not affect plant morphology under normal growth temperatures (25° C.) but did produce increased cytokinin concentrations and delayed leaf senescence in cold induced shoots.

We introduced cor15a-regulated genes (IPT and/or FAD7) into Nicotiana tabacum (cv. Havana), Dendranthema×grandiflorum (cv. Iridon), Nicotiana alata (flowering tobacco), and Petunia Hybrida (Marco polo Odyssey).

Enhanced Tolerance to Both Cold and Dark Conditions.

Double transgenic Nicotiana alata seedlings were generated, by crossing cor15a-FAD7 and cor15a-IPT parent lines, and the tolerance of the double transgenic seedlings to exposure to both dark and cold conditions was evaluated. RT-PCR analysis confirmed cold-induced expression of both the IPT and FAD7 genes putative double transgenic seedlings (FIG. 10). No IPT expression was observed in either line #33 or line #41 in the absence of a cold-induction signal. No FAD7 expression was observed in line #33 in the absence of a cold-induction signal, but a low level of expression was observed in line #41.

Both double transgenic T₁ generation lines #33 and #41 resisted injury under prolonged cold, dark conditions. On average, survival of double transgenic Nicotiana alata plants was dramatically higher (90% for line #33 and 89% for line #41) than for wild type plants (2%) following prolonged exposure to cold, dark conditions (P≦0.001).

Fatty Acid Analysis

Fatty acid analysis of Nicotiana alata leaves revealed that 16:0, 16:3, 18:2, and 18:3 were the major fatty acid species detected (Table 5) in wild type plants, while 16:0, 16:3 and 18:3 were the major fatty acid species detected in the double transgenic lines. The level of 18:3 was higher in the double transgenic (lines 41 and 33) plants than in wild type regardless of cold induction treatment (P≦0.01 under non-inductive conditions and P≦0.001 under cold inductive conditions). In response to cold-induction temperatures, 18:3 in wild type plants showed a marginal decline (P=0.08) and the 16:0 content showed a marginal increase (P=0.08). In the double transgenic lines, the 18:3 content remained stable after exposure to cold inductive temperatures and 16:3 increased 60% in line #41 (P≦0.05) and 49% in line #33 (P≦0.01), and the level of 16:0 decreased in both lines (P≦0.05). The fatty acid species 18:2 was detected only in wild type plants and was unaffected by cold inductive conditions.

Cold-Inducible Gene Expression

Reverse transcription-PCR (RT-PCR) analysis wild type and selected transgenic tobacco lines revealed a strong FAD7 gene transcription signal in cor15a-FAD7 tobacco exposed to the 4° C. cold-induction treatment but the transcription signal was very weak in cor15a-FAD7 tobacco plants (line N2) that were not exposed to the cold-induction treatment (FIG. 7). Wild type plants showed no evidence of FAD7 gene expression regardless of temperature treatment. These data demonstrate that FAD7 expression in cor15a-FAD7 plants could be dramatically up regulated via a cold-induction signal.

Cold Tolerance and Survival Rate of Wild Type and cor15a-FAD7 Transgenic Plants

Although short-term cold-induction treatment (4 days at 4° C.) caused no visible injury in cor15a-FAD7 transgenic or wild type tobacco seedlings, long-term exposure to cold (0.5 to 3.5° C.) caused visible injury and dramatically reduced survival in wild type plants. Chill injury first appeared as chlorosis and the degree and extent of injury became progressively more pronounced as exposure to the cold continued. After extended exposure to cold, most leaves on the transgenic cor15a-FAD7 remained green and the plants appeared healthy, but in contrast, wild type plants exhibited leaf damage, whole plant loss of turgor, shoot collapse and death.

After 44 days exposure to 0.5° C., 2° C., or 3.5° C., tobacco seedlings carrying the cor15a-FAD7 construct were more resistant to injury than the wild type and enjoyed dramatically higher survival rates (P≦0.001). Survival rate for wild type plants averaged 8.3% (averaged over the entire temperature range), while average survival for individual transgenic lines ranged from 54 to 79% (FIG. 8.). Survival rate varied with storage temperature (P≦0.001), with the highest survival rate observed at 2° C. for the wild type and all cor15a-FAD7 lines except N1 (which survived best at 3.5° C.). At 2° C. the survival rate of the wild type plants averaged 10.2% while the survival rate of cor15a-FAD7 lines N2 and N3 both averaged 96%.

Fatty Acid Composition in Leaves of Transgenic Plants

The most abundant fatty acids detected in the leaves of wild type N. tabacum grown at ambient temperatures were 16:0, 16:3, 18:2, and 18:3 (Table 4). In wild type plants exposed to cold treatment, there was a decline in the trienoic species, 16:3 (P≦0.05) and 18:3 (P≦0.05) and an increase in the dienoic species, 18:2 (P<0.05) and the saturated species, 18:0 (P<0.001). The change observed in the other major fatty acid species, 16:0 was not significant (P=0.07). Before exposure to cold induction, the fatty acid profile of the wild type and the FAD7 transgenic plants were similar with the exception of 18:0 (P<0.05) which was 2.5-times higher in the FAD7 plants. However after cold induction 16:3 and 18:3 were both higher in the FAD7 transgenic plants than in wild type plants (P≦0.01 and P≦0.001, respectively), and 18:0, 18:1 and 18:2 were all lower in the FAD7 transgenic plants than in wild type plants (P≦0.01, P≦0.001 and P≦0.001, respectively). In wild type plants exposed to cold induction the 16:3 levels declined 79% (P<0.05) while remaining stable in the FAD7 line N2 plants. In contrast, the level of 18:3 in wild type plants declined 20.6% after exposure to cold but increased 18.5% in FAD7 plants after exposure to cold. Also, after cold induction 18:2 increased by 58% in wild type plants (P≦0.05) but declined to non-detectable levels in FAD7 plants.

Effects of Prolonged Exposure to 4° C. on Chloroplast Ultrastructure and Chlorophyll Concentration

Thylakoid structure and organization appeared similar in chloroplasts from both wild type and cor15a-FAD7 transgenic prior to exposure to prolonged cold stress conditions (FIG. 9-I). However in wild type plants, exposure to cold stress resulted in extensive changes in chloroplast ultrastructure (FIG. 9-II a, b). After prolonged and continuous exposure to cold temperatures, micrographs of chloroplast from wild type plants revealed swelling, loss of granal stacking, and membrane disorganization typically associated with chloroplast death (FIG. 9-II a, b). In contrast, micrographs from cor15a-FAD7 leaves revealed that chloroplasts retained normal thylakoid structure and organization even after 40 days at 4° C. (FIG. 9-I c, d; FIG. 9-II c, d).

Changes in leaf chlorophyll concentration coincided with changes in chloroplast ultrastructure. Prior to exposure to cold, leaf chlorophyll concentrations were initially similar in both wild type and cor15a-FAD7 plants (919 and 916 ug/g fresh weight respectively). However after 40 days of exposure to cold (4° C.) leaf chlorophyll concentrations were dramatically lower (P≦0.001) in surviving wild type plants (144 ug/g FW) than in the cor15a-FAD7 plants (677 ug/g FW). Compared to the concentrations observed prior to cold, chlorophyll concentrations declined by 80% in wild type plants but only declined 28% in cor15a-FAD7 plants exposed to the same conditions.

Molecular Analysis of Transgenic Plants Expressing cor15a-ipt

Reverse transcription-PCR (RT-PCR) analysis was used to confirm ipt expression in transgenic lines in response to cold-induction signal. Total RNAs were extracted from the leaves of wild type and selected transgenic lines of chrysanthemum (lines 9 and 12) and petunia (lines 7 and 9) that were grown under normal conditions or first exposed to a 3-day cold-induction (4° C.) treatment. RT-PCR analysis showed that the 0.52 kb ipt DNA fragment was amplified in both cor15a-ipt chrysanthemum (line 9) and cor15a-ipt petunia (line 7) exposed to the 4° C. treatment but not in the same lines grown at the 25° C. and not exposed to the cold-induction treatment (FIG. 2). Similar results were obtained with line 9 of petunia and 12 line of chrysanthemum (data not shown). Wild type plants showed no evidence of ipt gene expression regardless of temperature treatment. These data demonstrate that ipt expression in cor15a-ipt plants could be up regulated with a cold-induction signal but remained suppressed at normal growing temperatures.

Leaf Senescence

The leaf senescence response of chrysanthemum and petunia under long-term dark storage conditions differed markedly between cor15a-ipt and wild type plants (FIG. 3). Overall, leaves from cold-induced cor15a-ipt plants remained green and healthy in prolonged dark storage while leaves from non-induced cor15a-ipt plants and from wild type plants, regardless of cold-induction treatments, did not. Similar results were observed with excised leaves of both chrysanthemum and petunia, and excised shoots and whole intact plants of chrysanthemum. For example, excised leaves of both wild type chrysanthemum and wild type petunia showed a dramatic loss of chlorophyll and advanced tissue senescence after 28 days in continuous darkness at 25° C. A pre-treatment of cold-induction temperatures had little effect on the course of tissue senescence under these conditions. However, when excised leaves of cor15a-ipt chrysanthemum and cor15a-ipt petunia were exposed to a cold-induction treatment (4° C. for 3 days) and then stored in the dark for 28 days, the tissue showed little or no visible symptoms of chlorophyll loss or tissue senescence. Leaves of cor15a-ipt chrysanthemum and cor15a-ipt petunia that were not exposed to a cold-induction treatment prior to dark storage developed symptoms of chlorophyll loss or tissue senescence that approached those observed in wild type leaves. With both excised shoots of cor15a-ipt chrysanthemum and whole intact cor15a-ipt chrysanthemum plants, a cold-induction treatment was required to produce the delayed onset of leaf senescence response under prolonged dark storage conditions.

Quantitative analysis revealed that leaf chlorophyll concentrations in cor15a-ipt petunia lines and wild type plants were similar under normal glasshouse growing conditions and showed a similar decline under prolonged dark storage conditions (FIG. 4). However, when plants were first exposed to a cold-induction treatment, the chlorophyll concentration in the cor15a-ipt lines remained at the level of normal grown plants even when exposed to prolonged dark storage. Cold induction had no beneficial effect on chlorophyll stability in the wild type plants and chlorophyll concentrations showed a precipitous decline in response to dark storage. Experiments with excised leaves of wild type and cor-15a-ipt chrysanthemum produced a similar response (data not shown).

Changes in Endogenous Concentrations of Cytokinins

Analysis of endogenous cytokinins, in freeze dried petunia shoot tips from cor15a-ipt plants, revealed a dramatic increase after a cold-induction treatment compared to concentrations from wild type plants (Table 1). Expression of the cor15a-ipt gene in petunia especially affected zeatin and dihydrozeatin type cytokinins. In cor15a-ipt plants, exposure to a cold-induction period (3.5 days at 4° C.) resulted in the increase of the physiologically active cytokinin trans-zeatin and its riboside (>4-fold and >18-fold, respectively), as well as of the storage cytokinins zeatin nucleotide (>10-fold increase), zeatin O-glucoside (5-fold increase) and the cytokinin deactivation products zeatin 7-glucoside (>10-fold increase) and zeatin-9-glucoside (>7-fold increase). The dihydrozeatin type cytokinins followed the same trend, but the increase was less dramatic. From the isopentenyladenine type cytokinins only the level of the active base (isopentenyladenine) was slightly increased during ipt expression at 4° C. The concentration of isopentenyladenosine was considerably elevated under growth permissive conditions (25° C.) in both wild type and transformed plants.

In cor15a-ipt chrysanthemum plants, cold-induced ipt gene expression produced marked increases in both the storage cytokinin pool (P≦0.05) and the pool of physiologically active cytokinins (P≦0.05), but the total cytokinin pool (active, deactivated and storage forms combined) was not substantially altered (Table 2). In more detail, trans-zeatin concentrations were similar in wild type chrysanthemum, under both non-inducing (25° C.) and cold-inducing (4° C.) temperatures, and in non-induced cor15a-ipt plants (averaging 8.2 pmol/g DW). However, concentrations increased (P≦0.05) in cor15a-ipt plants exposed to a prolonged period (14 d) at 4° C. and those exposed to a short cold induction period (3.5 d) followed by the transfer to the 25° C. growth permissive conditions for 3.5 d (averaging 16.8 pmol/g DW). But, 10.5 d after transfer to 25° C. the concentration of trans-zeatin in cold induced cor15a-ipt plants decreased to non-induced concentrations. In cor15a-ipt plants exposed to 4° C. for either 7 d or for 3.5 d followed by 3.5 d at 25° C., the concentration of trans-zeatin riboside (averaging 6.6 pmol/g DW) was measurably higher (P≦0.05) than in non-induced cor15a-ipt plants and wild type plants in both inductive and non-inductive conditions (averaging 1.5 pmol/g DW). The concentration of isopentenyladenine detected in non-induced wild type, non-induced cor15a-ipt, and cold induced wild type plants (4.1 pmol/g DW), was higher (P≦0.05) than the concentration found in cor15a-ipt plants exposed to cold for between 3.5 d and 14 d, or in plants exposed to cold for 3.5 d and then returned to 25° C. for 3.5 d (2.7 pmol/g DW). In contrast, the concentration of the corresponding riboside (iP7R) significantly increased (P≦0.05) in cor15a-ipt plants induced in cold for 3.5 d and then returned to growth conditions for either 3.5 d or 10.5 d (13.1 pmol/g DW) compared to the average concentration found in cold-induced wild type plants, non-induced wild type plants and non-induced cor15a-ipt plants (7.1 pmol/g DW). The concentration of iP7R was dramatically lower (P≦0.01) in cor15a-ipt plants after 3.5 d of cold-induction than when similar plants were transferred to growth conditions for 3.5 d or 10.5 d. The concentration of dihydrozeatin was low in all plants held at 4° C., but the concentration of dihydrozeatin riboside increased with ipt expression.

Plant Morphology

The overall growth habit of cor15a-ipt plants under growth chamber conditions (25° C.) was not substantially different from the wild type chrysanthemum line (Table 3). In addition, the overall growth response of both cor15a-ipt lines and wild type plants that were first exposed to a cold-induction treatment remained similar, indicating that the increase in ipt expression in cold-induced plants did not have a long lasting effect on subsequent plant growth. Of the growth parameters observed only shoot fresh weight and average lateral shoot length were affected by genotype. Shoot fresh weight for cor15a-ipt line #12 was similar to the wild type while shoot fresh weight for cor15a-ipt line #9 was lower. However, average lateral shoot length for cor15a-ipt line #12 was greater than either the wild type or cor15-ipt line #9. Shoot fresh weight and average leaf size (on the upper most lateral branch) were both affected by cold-induction treatment but both the cor15a-ipt lines and the wild type plants responded in the same way to this treatment. Most significantly there was no interactive effect of genotype and environmental treatment on any of the growth responses observed, indicating that any increase in cytokinin that resulted from a cold-induction period did not persist during plant development at normal glasshouse temperatures. Average number of lateral branches on each plant, number of secondary branches on each lateral shoot and average internode length on the top lateral branch were all unaffected by genotype or temperature treatment.

No differences were observed between non-induced wild type and cor15a-ipt petunia lines grown in the 25° C. growth chamber. For example, the average length of the main stem of non-induced wild type [21 cm (standard error 3.0)] and cor15a-ipt petunia plants [20.1 cm (se 0.6)] were similar. Likewise, the average number of lateral shoots on the main stem [5.8 (se 1.2) and 6.8 (se 0.4)], and the average internode length on the main stem [1.7 cm (se 0.3) and 1.5 cm (se 0.1)] were also similar for wild type and cor15a-ipt petunia plants, respectively. Even when exposed to an initial cold treatment, growth response was similar for the wild type and the cor15a-ipt transgenic petunia lines for four of the six parameters measured (length of the main stem, number of leaves on the main shoot, leaf area on the main shoot, and number of lateral branches on the main shoot). Compared to the wild type, shorter internodes were observed on two of the three transgenic lines tested and one transgenic line displayed a leaf area increase on the first lateral shoot. None of these anatomical features were consistent with the type of changes associated with constitutive ipt gene expression.

The present invention provides an isolated polynucleotide comprising a nucleic acid that encodes an isopentyltransferase (IPT) and/or a fatty acid desaturase (FAD7) and a nucleic acid encoding a heterologous promoter. The encoding nucleic acid is a fusion of the IPT gene or the FAD7 gene and the promoter gene, preferably in a construct where both genes are in an open reading frame such that when IPT and/or the FAD7 polypeptide is expressed in a plant, stimulation of one or more plant cytokinins occurs and membrane fatty acid desaturation occurs (increasing the number of double bonds in membrane fatty acids).

The IPT gene, ipt, employed will be an entire gene, such as a bacterial gene, preferably from Agrobacterium tumefaciens. The FAD7 gene, fad7, employed will be an entire gene, such as a plant gene, preferably from Arabidopsis thaliana. Of course due to the degeneracy of the genetic code, there are numerous replacement codons that will provide a particular amino acid and thus many substitutions can be made in a coding region without changing the primary structure of the encoded polypeptide. Additionally, it is contemplated that one may “plantize” ipt codons; i.e., substitute preferred amino acid codons in order to optimize expression for a particular plant species. Certain other substitutions in the coding region may also be made, such as those that substitute like amino acids; e.g., replacing one neutral amino acid codon with another neutral amino acid codon.

Likewise, mutant ipt and fad7, or truncated ipt and fad7 genes are also useful, so long as the chimeric fusion with a heterologous promoter gene is capable of being transferred into a plant cell and the encoded IPT or FAD7 expressed in the plant.

The ipt gene need not have a sequence identical to the bacterial ipt employed to illustrate the invention. So long as substantially similar activity is present and the construct is capable of stimulating endogenous cytokinin production in a transformed plant, the ipt nucleic acid sequence can be 80%, 90%, 95% up to 99% identical to the exemplary ipt.

Likewise, the fad7 gene gene need not have a sequence identical to the plant fad7 gene employed to illustrate the invention. So long as substantially similar activity is present and the construct is capable of stimulating endogenous fatty acid production in a transformed plant, the fad7 nucleic acid sequence can be 80%, 90%, 95% up to 99% identical to the exemplary fad7.

An important aspect of the invention is the heterologous promoter employed to construct the fusion gene. For illustration, the cor15a promoter fragment from the cor15a-gene was fused to ipt gene. In our study, the cor15a gene promoter was selected to drive gene expression so that an increase in fatty acid desaturation or cytokinin production would occur after the plants were exposed to a brief but specific environmental signal. The cor15a gene is a member of the COR (cold-regulated) gene family. Arabidopsis cor15a gene is cold regulated, has CRT/DRE regulatory elements, and is induced in response to the CBF transcriptional activators (Thomashow 1999; Thomashow 2001; Jaglo et al. 2001). Cor15a is inactive, or very weakly active, in most plant tissues and plant organs maintained under normal grown temperatures but becomes highly active in plant shoots in response to low temperature (Baker et al. 1994).

The invention also comprises expression vectors harboring the chimeric genes constructed from an IPT-encoding nucleic acid, e.g. SEQ ID NO:8 and a heterologous promoter such as an cor15a promoter (SEQ ID NO:7), or genes constructed from an FAD-encoding nucleic acid, e.g. SEQ ID NO:9 and a heterologous promoter such as an cor15a promoter (SEQ ID NO:7). Particular examples of promoters are those encoded by the nucleic acid sequences of SEQ ID NO:7. The ipt gene would be expected to have similar effect at identities of at least 80-90% of SEQ ID NO:8. The fad7 gene would be expected to have similar effect at identities of at least 80-90% of SEQ ID NO:9.

In other aspects of the invention, a host plant cell comprising the described expression vectors is contemplated, including transgenic plants propagated from such plant cells. Plants from which cells may be obtained or used for transformations include any horticultural or agronomic crop that uses seedlings or rooted cuttings as transplants at the onset of the production cycle, or that requires prolonged cold storage, or dark and cold storage during the post-harvest phase of production. In the examples herein, we utilized tobacco, chrysanthemum, petunia, and flowering tobacco as representative crop species.

In particular embodiments, the invention also includes an isolated nucleic acid sequence comprising the nucleic acid sequence selected from the group consisting of SEQ ID NO: 7 in open reading frame with a nucleic acid encoding a heterologous isopentenyl transferase (IPT).

Another embodiment is an isolated nucleic acid sequence encoding isopentenyl transferase (IPT) fused 5′ with an cor15a promoter capable of expressing the IPT in a plant. More particularly, the nucleic acid sequence is SEQ ID NO:8 fused with a nucleic acid having the sequence of SEQ ID NO: 7 (cor15a promoter).

In particular embodiments, the invention also includes an isolated nucleic acid sequence comprising the nucleic acid sequence selected from the group consisting of SEQ ID NO: 7 in open reading frame with a nucleic acid encoding a heterologous fatty acid desaturase (FAD7).

Another embodiment is an isolated nucleic acid sequence encoding fatty acid desaturase (FAD7) fused 5′ with a cor15a promoter capable of expressing the FAD7 in a plant. More particularly, the nucleic acid sequence is SEQ ID NO:9 fused with a nucleic acid having the sequence of SEQ ID NO: 7 (cor15a promoter).

Transgenic plants transformed with the disclosed cor15a-ipt and/or the cor15a-fad7 constructs are a particularly important aspect of the invention. These transformed plants exhibit significantly increased tolerance to prolonged dark & cold, or just cold storage compared to the untransformed plants of the same species. Particularly significant results have been demonstrated with tobacco, chrysanthemum, petunia, and flowering tobacco and are expected to be similar in other important horticultural and agronomic species that uses seedlings or rooted cuttings as transplants at the onset of the production cycle, or that require prolonged cold storage, or dark and cold storage during the post-harvest phase of production.

Exemplary families include the Cruciferae, Cucubitaceae, Compositae, Solanaceae, Euphorbiaceae, Gramineae, Leguminosae, and Rosaceae. In the Rosaceae there are over 3200 species with many important horticultural species such as plums, peaches, cherries, apricots & almonds in the genus Prunus. In the Compositae, there are over 20,000 species including many popular ornamental species such as chyrsanthemum, asters, sunflower, marigold and the like. The Gramineae family includes the worlds most important grain crops including rice, wheat, oats, barley, rye, and maize. It also includes sugar cane, important structural species such as bamboo, forages used for animal feeds and many important ornamental and turf species.

The invention also includes progeny of the described transgenic plants and seeds from these plants. The progeny show the same enhanced phenotypic traits as the parent. DNA analysis shows that one or more copies of the transgene can be incorporated into plant genome. Depending on the position of incorporation, the phenotypes; e.g., tolerance to prolonged cold storage, or dark and cold storage, as transplants or during the post-harvest phase of production, may vary in different plants transformed with the same expression vector; however, it is believed that vegetative propagation of a plant with highly desirable phenotype will result in substantially identical clones, allowing elimination of plants that do not have marketable appearance or productivity.

Related aspects of the invention include methods for increasing endogenous cytokinin levels in a plant, comprising transforming a plant with a transgene comprising an isopentenyltransferase (IPT)-encoding nucleic acid fused with an cor15a-encoding nucleic acid. Expression of the transgene in the plant causes increased cytokinin levels in the transformed compared with a non-transformed plant of the same species. The increased cytokinin levels modify normal senescence characteristics during dark storage but not during active growth in the production environment.

Similarly, the invention include methods for altering membrane fatty acid composition in a plant, comprising transforming a plant with a transgene comprising a fatty acid desaturase (FAD)-encoding nucleic acid fused with an cor15a-encoding nucleic acid. Expression of the transgene in the plant causes increased increased tolerance to cold temperatures in the transformed compared with a non-transformed plant of the same species. The increased number of double bonds in membrane fatty acids modifies normal cold-tolerance characteristics of cell membranes during cold storage but not during active growth in the production environment.

In the transgenic plant harboring both trait genes, increase tolerance to both cold, and dark storage conditions will result. These traits will result in readily detectable changes the length of time leaves remain green, the length of time plants and plant parts in cold storage remain health and viable when compared with untransformed plants of the same species. Particularly suitable plants for transformation important horticultural and agronomic species that utilize seedlings or rooted cuttings as transplants at the onset of the production cycle, or that require prolonged storage in cold, or cold and dark conditions, during the post-harvest period such as during shipment to market.

Preferred cold-regulated (cor) promoters for use in constructing the described chimeric genes include cor15a such as promoters having the nucleic acid sequence of SEQ ID NO:7.

The methods described for the transgenic plants of the invention will have increased levels of desaturated fatty acids in cell membranes where the regulation of fatty acid desaturation can be affected by expression of the fatty acid desaturase gene under control of an cold-regulated gene promoter fragment.

Further, the methods described for the transgenic plants of the invention may also have increased levels of cytokinins where endogenous production of these cytokinins can be affected by expression of the isopentenyltransferase gene under control of an cold-regulated gene promoter fragment.

Overall and in general therefore, the invention includes expression vectors comprising any of the described fusion polypolypeptides encoded by a chimeric gene constructed from an ipt gene, and a fad7 gene, and a cor15a promoter sequence. Also included are host plant cells and transgenic plants harboring the disclosed expression vectors.

For commercial purposes, and of use to growers who may wish to further develop plants in the manner disclosed, a packaged kit is contemplated. Such a kit may include both a cor15a-ipt and a cor15a fad7 chimera comprised within suitable transfection plasmids and directions for use of the plasmids suitable for various plant species.

### BRIEF DESCRIPTION OF THE FIGURES

FIG. 1. A, B & C. PCR analysis of DNA from putative transgenic chrysanthemum lines and Southern blot analysis of genomic DNA isolated from petunia plants. (A) PCR analysis showing the presence of the expected 0.98 kb fragment of the cor15a promoter from putative-transgenic chrysanthemum lines. Lane 1: 1 kb molecular marker; Lane 2: Negative control (untransformed chrysanthemum plant); Lane 3: Positive control (plasmid cor15a-ipt-nos); Lanes 4-8: putative-transgenic chrysanthemum plants. (B) PCR analysis showing the presence of the expected 0.52 kb fragment of ipt gene from putative-transgenic chrysanthemum lines. Lane 1: 1 kb molecular mark; Lane 2: Positive control (plasmid cor15a-ipt-nos); Lane 3-7: Transgenic chrysanthemum plants; Lane 8: Negative control (untransformed chrysanthemum plant). (C) Southern blot analysis of genomic DNA from cor15a-ipt petunia lines. A DIG-labeled 0.523 kb fragment of the ipt gene from the cor15a-ipt-nos plasmid DNA was used as a probe. An ipt fragment was detected from each of the transgenic lines analyzed (Lanes 1-3) but not from wild type petunia DNA (Lane 4-5). Plasmid DNA was used as the positive control (Lane 6).

FIG. 2. RT-PCR analysis of ipt expression in cor15a-ipt and wild type chrysanthemum (A) and petunia (B) plants exposed to normal growing temperatures or a cold-induction treatment. Lanes: 1—wild type plant (normal conditions, 25° C.), 2—wild type plant exposed to a cold-induction treatment (3.5 d at 4° C.), 3—transgenic plant (normal conditions, 25° C.), 4—transgenic plant exposed to a cold-induction treatment (3.5 d at 4° C.).

FIG. 3. Senescence in excised leaves, stems and whole plants of wild type and cor15a-ipt plants. (Row 1): Excised cor15a-ipt and wild type chrysanthemum leaves were either exposed to cold-inducing or non-inducing temperatures prior to dark storage. Leaves from cor15a-ipt plants that did not receive a cold-induction treatment prior to storage senesced (A) while leaves that received a cold-induction treatment prior to storage remained green (B). Wild type chrysanthemum leaves senesced in dark storage whether first exposed to cold-temperatures (C) or not (D). (Row 2): Similarly, cor15a-ipt petunia leaves that did not receive a cold-induction treatment prior to storage senesced (E) while leaves that received a cold-induction treatment prior to storage remained green (F). Wild type petunia leaves senesced in dark storage whether first exposed to cold temperatures (H) or not (G). (Row 3): Excised chrysanthemum shoots from wild type and cor15a-ipt chrysanthemum were either exposed to cold (3 days at 4° C.) or non-inducing (25° C.) temperatures prior to 18 days dark storage at 25° C. Shoots from wild type plants senesced when stored in the dark regardless of cold induction treatment (I=no cold induction; J=cold induced). Shoots from cor15a-ipt plants stored without cold-induction also senesced in dark storage (K), but shoots stored after exposure to cold-induction temperatures remained healthy (L). (Row 4): Intact cor15a-ipt chrysanthemum plants after 24 days of incubation in dark conditions. Transgenic plants that were not exposed to cold prior to dark storage senesced (M) but plants that were exposed to a cold-induction period (3.5 d at 4° C.) prior to dark storage did not (N).

FIG. 4. Chlorophyll concentrations in leaves of wild type (control) and cor15a-ipt petunia plants (from lines 7 and 9) under growth conditions (in the glasshouse), or following 14 d dark storage without prior cold-induction, or exposed to a cold-induction treatment (3 d at 4° C.) prior to 14 d dark storage. Each value represents the mean of seven observations. Error bars represent the standard error of means.

Table 1. Cytokinin concentrations in wild type and cor15a-ipt transgenic petunia plants exposed to cold-induction or non-inducing conditions. Values represent the mean of samples from three different plants from the same transgenic line. Z, trans-zeatin; ZR, trans-zeatin 9-riboside; ZRMP, trans-zeatin 9-riboside-5′-monophosphate; ZOG, trans-zeatin O-glucoside; Z7G, trans-zeatin 7-glucoside; Z9G, trans-zeatin 9-glucoside; DHZ, dihydrozeatin; DHZR, dihydrozeatin 9-riboside; DHZROG, dihydrozeatin 9-riboside O-glucoside; iP, N⁶-(Δ²-isopenenyl)adenine; iPR, N⁶-(Δ²-isopentenyl)adenosine; iPRMP, N⁶-(Δ²-isopentenyl) 9-riboside-5′-monophosphate.

Table 2. Concentrations of physiologically active cytokinins (trans-zeatin, isopentenyladenine, dihydrozeatin and the corresponding ribosides), O-glucosides (of trans-zeatin, trans-zeatin riboside and dihydrozeatin riboside) and total cytokinins in leaves of wild type and cor15a-ipt chrysanthemum plants exposed to different inductive and non-inductive temperature conditions prior to sampling.

Table 3. Growth characteristics of wild type and cor15a-ipt transgenic chrysanthemums. Plants were grown in the vegetative state in the growth chamber under 25° C. day (16 h) and 20° C. night temperature conditions. Half the plants received a cold-induction treatment (3 d at 4° C.) and 1 week 25° C. dark storage prior to the growth study and the remaining plants were not exposed to cold induction temperatures.

Table 4. Fatty acid profile from leaves of wild type (WT) and transgenic lines of Nicotiana tabacum (line N2) containing the fatty acid desaturase (FAD7) gene under the control of a cold-inducible promoter (cor15a). The major fatty acid components were isolated from total lipids extracted from young mature leaves. Leaf samples were obtained from plants grown under both normal greenhouse conditions (25° C.) and from similar plants after exposure to cold-inductive temperatures (4° C.). Each value represents the mean of three independent experiments.

Table 5. Fatty acid profile from leaves of wild type (WT) and double transgenic Nicotiana alata (lines #33 & #41) containing both the FAD7 and IPT genes under the control of a cold inducible promoter (cor15a). The major fatty acid components were isolated from total lipids extracted from young mature leaves. Leaf samples were obtained from plants grown under both normal greenhouse conditions (non-inducing temperatures) and from similar plants after exposure to cold-inductive temperatures. Each value represents the mean of three independent experiments.

FIG. 5. Scheme showing the structure of the cor15a-FAD7-nos construct. From left to right: RB-right border of pBin19; P-nos-nopaline synthase promoter; NPTII—neomycin phosphotransferase (nptII) gene from Tn5; T-nos—nopaline synthase terminator; cor15a—promoter from the cor15a gene; FAD7—gene from Arabidopsis thaliana; T-nos—nopaline synthase terminator; LB—left border of pBin19.

FIG. 6. PCR analysis of DNA from putative transgenic tobacco lines and Southern blot analysis of genomic DNA isolated from T₂ generation of tobacco plants. (a) PCR analysis showing the presence of the expected 0.98 kb fragment of the cor15a promoter from putative-transgenic tobacco lines. Lane 1: 1 kb molecular marker; Lane 2: Positive control (plasmid cor15a-FAD7-nos); Lane 3: Negative control (untransformed tobacco plants); Lanes 4-8: putative-transgenic tobacco plants: (b) PCR analysis showing the presence of the expected 1.25 kb fragment of FAD7 gene from putative-transgenic tobacco lines. Lane 1: 1 kb molecular marker; Lane 2: Positive control (plasmid cor15a-FAD7-nos); Lane 3: Negative control (untransformed tobacco plants); Lane 4-8: putative-transgenic tobacco plants. (c) Southern blot analysis of genomic DNA from cor15a-FAD7 tobacco plants (generation T₂). A DIG-labeled fragment of the FAD7 gene from the cor15a-FAD7-nos plasmid DNA was used as a probe. An FAD7 fragment was detected from each of the transgenic lines analyzed (Lanes 1-3) but not from wild-type tobacco DNA (Lane 4). Plasmid DNA was used as the positive control (Lane 5).

FIG. 7. RT-PCR analysis of FAD7 expression in cor15a-FAD7 and wild type Nicotiana tabacum plants exposed to normal growing temperatures (non-inductive conditions) or a cold-induction treatment (4° C. for 3 days). Lanes: 1—wild type (control) tobacco under non-inductive conditions, 2—wild type tobacco after 3 days exposure to a cold-induction treatment, 3—cor15a-FAD7 transgenic tobacco under non-inductive conditions, 4—cor15a-FAD7 transgenic tobacco after 3 days of a cold-induction treatment.

FIG. 8. Young seedlings of wild type (WT) and six independent cor15a-FAD7 transgenic tobacco lines after 44 days in cold storage at 0.5, 2 or 3.5° C. Over the entire temperature range survival of wild type plants averaged just 8.3% but survival of individual cor15a-FAD7 transgenic tobacco lines was dramatically higher (P<0.001). Bars represent standard error (SE) of the mean survival rate.

FIG. 9. Transmission electron micrographs of chloroplast ultrastructure in wild-type (a, b) and cor15a-FAD7 transgenic tobacco leaves (c, d) under normal greenhouse conditions (I) and after 40 days at 4° C. (II). I-Prior to exposure to cold stress conditions the chloroplast ultrastructure appeared similar in micrographs obtained from both wild type (a and b) and cor15a-FAD7 (c and d) plants. II-After prolonged and continuous exposure to cold temperatures, micrographs of chloroplast from wild type plants revealed.

FIG. 10. RT-PCR analysis of FAD7 (a) and IPT (b) gene expression in double transgenic Nicotiana alata lines 33 and 41 containing both genes under the control of a cold-inducible (cor15a) promoter. Gene expression was assessed in plants exposed to both non-inductive temperatures (normal growing temperatures) and cold-inductive temperatures. For both a and b, lanes: 1—double transgenic line #33 under non-inductive temperatures (25° C.), 2—double transgenic line #41 under non-inductive temperatures (25° C.), 3—double transgenic line #33 exposed to cold-inductive temperatures (5 d at 4° C.), 4—double transgenic line #41 exposed to cold-inductive temperatures (5 d at 4° C.).

FIG. 11: SEQ ID NO:1. Primer used for DNA amplification of the 0.982 kb fragment of the cor15a gene promoter.

FIG. 12: SEQ ID NO:2. Primer used for DNA amplification of the 0.982 kb fragment of the cor15a gene promoter.

FIG. 13: SEQ ID NO:3. Primer used for DNA amplification of the 0.523 kb region of ipt gene.

FIG. 14: SEQ ID NO: 4. Primer used for DNA amplification of the 0.523 kb region of ipt gene.

FIG. 15: SEQ ID NO: 5. The primer used for DNA amplification of the 1.176 kb fragment of the FAD7 gene.

FIG. 16: SEQ ID NO: 6. The primer used for DNA amplification of the 1.176 kb fragment of the FAD7 gene.

FIG. 17: SEQ ID NO: 7. Base sequence for the promoter region of the cor15a gene.

FIG. 18: SEQ ID NO: 8. Base sequence for the coding region of the IPT gene.

FIG. 19: SEQ ID NO: 9.Base sequence for the coding region of the FAD7 gene.

FIG. 20: SEQ ID NO: 10. Full cor15a-IPT-nos sequence (CD enclosed).

FIG. 21: SEQ ID NO: 11. Full cor15a-FAD7-nos sequence (CD enclosed).

### DETAILED DESCRIPTION OF THE INVENTION

The present invention provides nucleic acids, vectors and expression cassettes that are capable of modifying both cytokinin production in plants and fatty acid composition in plant cellular membranes in response to a specific and controllable environmental signal (e.g. cold). These transgenic traits enhance certain desirable phenotypic characteristics such as increasing chlorophyll retention under dark conditions, and increasing membrane stability under cold conditions. The use of the cor15a-fad7 trait alone will produce greater cold-storage tolerance for post-harvest storage of plants parts (e.g. cut flowers, fruits & vegetables) while use of both trait genes (cor15a-ipt & cor15a-fad7) together will increase tolerance of young plants (e.g. seedlings and rooted cuttings) to withstand prolonged cold, dark storage and still remain viable when planted in the field or glasshouse.

These novel constructs are designed to express both the ipt gene product, isopentenyltransferase (IPT) and the fad7 gene produce (fatty acid desaturase), under specific environmental conditions that occur in cold storage but not under production conditions. Gene expression is achieved when the cor15a fragment of the cor15a gene promotor is activated. This is accomplished by engineering a chimera, comprising at least one cold-regulated gene promoter (such as cor15a) fused to an isopentenyltransferase (IPT)—encoding DNA or a fatty acid desaturase (FAD)—encoding DNA. When plant cells are transformed with an expression vector harboring these chimeric genes, activation of the cor15a promoter causes upregulation of the IPT gene producing expression of the isopentenyltransferase enzyme or upregulation of the FAD7 gene producing expression of the fatty acid desaturase enzyme. The result is increased production of endogenous cytokinin in response to the cold signal (in the case of cor15a-ipt; SEQ ID NO#: 10) and/or increased desaturation of membrane fatty acids in response to the cold signal (in the case of cor15a-fad7; SEQ ID NO#: 11) in the affected plant tissue.

In the horticulture trade, billions of plugs (e.g. whole plants used for transplant) and vegetative cuttings are produced annually for sale to commercial growers. Stockpiling plugs and cuttings for later use is advantageous because it increases productivity. However, long-term storage can only be successful if the majority of the plants survive and remain vigorous. Such storage requires cold temperatures to minimize respiration and the rapid deterioration of chlorophyll that results when plants are exposed to low light and warm temperatures (Heins et. al., 1995).

Herein we demonstrate control of ipt expression, and consequently delayed leaf senescence, under specific stress conditions using the cold-inducible promoter from the cor15a gene from Arabidopsis thaliana. This promoter was selected so that gene expression would occur only after the plants were exposed to a brief but specific environmental stress. The cor15a gene is a member of the COR (cold regulated) gene family. Cor15a encodes a 15-kDa polypeptide that is targeted to the chloroplasts. Upon transit into the organelle, the cor15a peptide is processed to a 9.4-kDa polypeptide designated as cor15am. The constitutive expression of cor15a in non-acclimated transgenic Arabidopsis plants increases the freezing tolerance of both chloroplasts frozen in situ and isolated leaf protoplasts frozen in vitro by 1 to 2° C. over the temperature range of −4 to −8° C. (Thomashow, 1999). Baker et al. (1994) showed that the cor15a promoter is inactive, or very weakly active, in most of the tissues and plant organs maintained under temperatures associated with active growth and, that in response to low temperature, it becomes highly activate in the shoots but not in the roots (Baker et al., 1994). Root expression of ipt is a concern with asexually propagated species because of the potential for cytokinins to impede root development. Analysis of the cis-elements within the cor15a promoter indicated that the 5′ region between nucleotides −305 and +78 imparted ABA- and drought-regulated gene expression in addition to cold-regulated expression (Baker et al., 1994). Therefore, to avoid undesirable stress, which could also affect ipt gene expression, we carried out all morphological experiments under carefully controlled environmental conditions.

In our experiments, RT-PCR analysis confirmed that ipt expression was a result of cold-activation and no transcript was detected in either wild type plants or transgenic plants that were not exposed to cold temperature conditions. Our results are in accordance with the data reported by Hajela et al. (1990), who detected cor transcripts (regulated by the cor15a promoter) 1 to 4 h after Arabidopsis plants were exposed to cold temperatures. The amount of transcripts continued to increase for about 12 h and then remained elevated as long as the plants remained in the cold (up to 14 days in their study). However, when the plants were returned to normal growth temperatures, transcripts decreased rapidly and returned to concentrations found in the non-transgenic plants after 8 h.

Our experiments showed that expression of ipt gene in petunia resulted in an immediate increase of zeatin and dihydrozeatin type cytokinins. We found 5.3-fold increase of physiologically active foams (trans-zeatin, dihydrozeatin and their ribosides), 3.6-fold of storage forms (O-glucosides of trans-zeatin and dihydrozeatin and trans-zeatin nucleotide) and 5.4-fold of deactivation forms (N-glucosides of trans-zeatin and dihydrozeatin). The increase in the concentration of isopentenyladenine, but not of its derivatives, indicates that cytokinin metabolism, including of the hydroxylation of the isoprenoid side chain, is very fast in this species. High concentrations of isopentenyladenosine under growth permissive conditions is in accordance with the results of Auer et al. (1999), who found an increase in isopentenyladenine/isopentenyladenosine in Petunia hybrida explants during shoot induction and especially in the shoot developmental phase. When considering the relatively moderate increase of endogenous cytokinins that followed cold-induced ipt expression, it is also necessary to consider that the increase in cytokinin biosynthesis probably stimulated additional cytokinin oxidase/dehydrogenase activity, in a way similar to what was detected in petunia after application of BA (Auer et al., 1999).

In chrysanthemum plants, ipt expression led to the accumulation of storage cytokinins (O-glucosides), and only after prolonged cold induction (more than 7 days), an increase in active cytokinins occurred. Short induction (3.5 d) followed by the plant transfer to growth permissive conditions (25° C.) resulted in marked increase of all physiologically active cytokinins, accompanied by the decrease of cytokinin O-glucosides. This difference between petunia and chrysanthemum plants may be due to specific differences in adaptation to cold temperatures. In chrysanthemum the concentration of active cytokinins seems to be tightly regulated in response to the temperature. A decrease in concentration of active cytokinin species during prolonged incubation at 25° C. would be expected if the storage temperature led to a decrease in transcript in conjunction with continued cytokinin turnover (affected by cytokinin oxidase/dehydrogenase).

In our study, an overall increase in cytokinin concentrations in cold-induced cor15a-ipt petunia and chrysanthemum plants did not reach the level reported after ipt overexpression in other systems. For example when ipt was placed under the control of a Drosophila heat-inducible promoter (hsp70) and introduced in Nicotiana plumbaginifolia, the resulting increase in cytokinin concentration ranged from 140- to 200-fold compared to non-induced leaves (Smigocki, 1991). The resulting transgenic plants were shorter, had underdeveloped root system and reduced leaf width. Another transgenic tobacco containing the maize hsp70-ipt gene exhibited an after-heat-treatment increase in zeatin and zeatin riboside concentrations of 52 and 23-fold, respectively (Medford et al., 1989). In these studies, a consistent, low level of expression was observed even under non-inducing conditions and plant phenotype was dramatically affected, especially at the higher cytokinin concentrations (Medford et al., 1989). When a more tightly regulated soybean heat-inducible promoter was used to regulate ipt expression, ipt transcription was not detected in plants exposed to normal temperatures and plants did not display the phenotypic characteristics associated with constant ipt expression. After heat shock, zeatin riboside concentration increased only 5-fold and the plants developed with shorted internodes, crinkled and down-folded leaves and enlarged stems. Transgenic plants also displayed delayed leaf senescence and flower bud development (Ainley et al., 1993). Still others have reported that a sharp, transient increase in cytokinins was sufficient to promote plant cell division (Redig et al., 1996, Dobrev et al., 2002), and even a temporary increase in cytokinin triggered changes in organ initiation and differentiation (Kaminek et al., 1997).

Regardless of the magnitude of changes in cytokinin concentrations observed in cor15a-ipt petunia and chrysanthemum lines, the cold-induced plants in our study displayed a dramatic increase in chlorophyll retention and a dramatic delay in senescence under warm, dark storage conditions. For example, following exposure to a 72 h activation period at 4° C., leaves from cor15a-ipt petunia and chrysanthemum remained healthy and green even after 3 weeks of dark incubation at 25° C. Leaves of non-transformed plants senesced under the same storage regime. Similar responses were observed for shoot tip cuttings and whole plants. In addition, actively growing cor15a-ipt lines exhibited growth and development characteristics that were similar to the wild type petunia and chrysanthemum phenotypes. A normal phenotype was observed even when cor15a-ipt lines were initially exposed to cold activation temperatures before growing in the 25° C. environment. Thus, up-regulation of cor15a-ipt in response to cold-induction appeared to be sufficient to alter leaf senescence properties of petunia and chrysanthemum but, under light and temperature conditions associated with active growth, the presence of the cor15a-ipt gene did not elicit the type of undesirable phenomic responses associated with constitutive ipt expression.

In our study, we tested a new genetic construct capable of regulating the degree of membrane fatty acid desaturation in response to a cold-induction signal. The FAD7 chloroplast co-3 fatty acid desaturase gene from Arabidopsis thaliana was cloned under the control of the cold-inducible promoter from the cor15a gene from Arabidopsis, and the construct was introduced into Nicotiana tabacum and Nicotiana alata. In both species, transgenic plants showed superior tolerance to prolonged exposure to cold temperatures. Tolerance was characterized by increased plant survival that coincided with increased retention of chlorophyll, and stability in both the membrane trienoic fatty acid components (16:3 and 18:3) and the chloroplast thylakoid ultrastructure.

The importance of membrane fluidity in temperature tolerance has been delineated by mutation analysis, transgenic, and physiological studies (Sung et al. 2003; Orvar et al. 2000). A change in membrane fluidity is one of the immediate consequences of exposure to low temperature, and in chilling-sensitive species, membrane lipids represent a potential site of temperature perception and/or injury. Although we do not report changes in membrane fluidity, this property is largely dictated by the composition of the lipid molecular species, the degree of membrane saturation and temperature.

The FAD7 gene catalyzes desaturation of the 16- and 18-carbon lipid-linked dienoic fatty acids (16:2 and 18:2). The amino-terminal region of the FAD7 gene product carries a chloroplast transit peptide so that the primary affect of FAD7 gene expression is on chloroplast membranes (Iba et al.1993). The FAD7 ω-3 fatty acid desaturase enzyme acts on 16- and 18-carbon fatty acids in either the sn-1 or sn-2 position of all chloroplast lipids including monogalactosyl diacylglycerol, digalactosyl diacylglycerol, phosphatidylglycerol, and sulfolipids. However Iba et al. (1993) reported that mutants deficient in FAD7 activity also experienced changes in extra-chloroplastic lipid composition. This phenomenon was attributed to a change in the trienoic fatty acid flux from the chloroplast to extra-chloroplastic membranes. Ishizaki-Nishizawa et al. (1996) reported that transgenic plants expressing a cyanobacterial desaturase gene also had a higher level of unsaturated fatty acid content in most membrane lipids and exhibited a significant increase in chill resistance. Kodama et al. (1994) expressed Arabidopsis desaturase FAD7 gene in tobacco controlled by the 35S promoter and observed a reduction in the incidence of low-temperature-induced chlorosis found in wild-type plants exposed to the same stress conditions. These studies conclusively demonstrated that resistance to injury from chilling could be increased by selectively expressing of a fatty acid desaturation gene.

In our study, the cor15a gene promoter was selected to drive FAD7 expression so that an increase in fatty acid desaturation would occur after the plants were exposed to a brief but specific environmental signal. The cor15a gene is a member of the COR (cold-regulated) gene family. Arabidopsis cor15a gene is cold regulated, has CRT/DRE regulatory elements, and is induced in response to the CBF transcriptional activators (Thomashow 1999; Thomashow 2001; Jaglo et al. 2001). Cor15a is inactive, or very weakly active, in most plant tissues and plant organs maintained under normal grown temperatures but becomes highly active in plant shoots in response to low temperature (Baker et al. 1994). Previously, Khodakovskaya et al. (2005) used the cor15a promoter to drive expression of the IPT gene (resulting in increased tissue cytokinin concentrations) in petunia and chrysanthemum, and demonstrated that IPT expression could be regulated in response to a short (3-d) cold treatment. In that study, IPT expression did not affect plant morphology under normal growth temperatures (25° C.) but did produce increased cytokinin concentrations and delayed leaf senescence in cold induced shoots.

Chilling tolerance in plants represents an important agronomic trait. Young transplants or vegetative cuttings that can survive prolonged cold storage and then resume vigorous growth allow for greater productivity and greater flexibility for the commercial propagator. A molecular strategy whereby transgenic plants selectively express greater thylakoid membrane stability under cold stress conditions represents a novel approach to selective chill-tolerance in plants. On the basis of this scenario, we constructed the cor15a-FAD7 fusion gene (SEQ ID NO#: 11) and tested the effects of this gene on membrane stability and long-term survival under cold conditions. Our data demonstrate that cold-induced expression of the trait gene for increased desaturation of chloroplastic membrane fatty acids effectively and dramatically increased seedling survival under prolonged cold storage and that greater thylakoid membrane stability was associated with survival.

In cor15a-FAD7 tobacco, RT-PCR analysis indicated that FAD7 expression dramatically increased in response of cold treatment (4° C. for 3 days), although some expression was noted at higher temperatures (25° C.). The most striking affect of increased desaturase gene expression in cold-induced cor15a-FAD7 plants was the increase survival under long-term cold storage. For example, survival of non-transgenic (wild-type) N. tabacum after 44 days in cold—storage (0.5° C., 2° C., 3.5° C.) averaged only 8.3%. However, survival of plants from individual cor15a-FAD7 lines ranged from 54 to 80% under the same conditions. Analyses of leaf fatty acid composition revealed that the trienoic fatty acids, 18:3 and 16:3 remained stable in cor15a-FAD7 transgenic N. tabacum after exposure to cold while the saturated fatty acids (16:0, 18:0) decreased (P≦0.01). In contrast, the proportion of 16:3 and 18:3 in wild-type plants, decreased after exposure to cold-stress conditions (P≦0.01 and P≦0.05, respectively) whereas the saturated fatty acid species 18:0 increased (P≦0.001). The proportion (mol %) of 18:2 increased by 58% in wild type plants while decreasing to a non-detectable level in the cor15a-FAD7 plants after exposure to cold. Kodama et al. (1994) reported a similar phenomenon in transgenic 35S-FAD7 tobacco seedlings with a decrease in 16:2 and 18:2 concomitant with an increase in the 16:3 and 18:3.

It may be that the decline in 16:3 and 18:3 in wild type plants is responsible for changes to the chloroplast ultrastructure that coincide with the loss of chlorophyll and poor survival under prolonged cold stress conditions. McConn and Browse (1996) used a FAD7-1 mutant that produced very low concentrations of trienoic acids (16:3 and 18:3) to demonstrate a clear correlation between the severity of chlorosis after exposure to cold (4° C. for 30 days) and the proportion of trienoic fatty acids in the leaves. Routaboul et al. (2000) used the triple mutant fad3-2 fad?-2 fad8 which has no detectable levels of trienoic acids to demonstrate a correlation in chlorophyll content to prolonged cold exposure. In our study, electron microscopy confirmed that changes in chloroplast ultrastructure coincided with the increased incidence of visible injury (e.g. chlorosis, necrosis, loss of turgor and death) in wild-type tobacco plants. After 40 days at 4° C., granal disorganization appeared in wild type plastids. In contrast, chloroplast membranes in cor15a-FAD7 plants appeared well organized and unaffected by the cold stress, and both chlorophyll retention and survival were substantially higher. Studies have shown that Arapidopsis thaliana fad5 mutants which are devoid of 16:3, but have wild type levels of 18:3, became chlorotic, and experience reduced growth and chloroplast ultrastructure changes similar to those seen in our study with wild type plants (FIG. 9-II a, b) exposed to cold for prolonged periods (Hugly et al. 1992). The cold effects seen in fad 5 plants were dependent on the leaf development stage (Hugly et al. 1992). Routaboul et al. (2000) also observed similar damage to the chloroplast ultrastructure when FAD7 deficient mutants were exposed to long-term cold storage (30 days at 4° C.). Thus, the use of the cold-inducible FAD7 gene has obvious potential for producing cold tolerant plants that selectively express the trait only in response to a cold signal.

In commercial horticulture, plants and excised plant parts are typically stored under cool, dark conditions but the incidence of chilling injury and mortality increases with storage duration (Heins et al. 1995). Therefore it is important to improve both cold and dark storage tolerance in sensitive species. In our study, we established transgenic Nicotiana alata plants that expressed two independent transgenes, IPT (isopentenyl transferase) and FAD that were both controlled by the cold-inducible cor15a promoter. Previously, Khodakovskaya et al., (2005) reported that cold-induced plants carrying the cor15a-IPT construct retained high chlorophyll concentrations, coincident with increased cytokinin concentrations, after prolonged exposure to dark conditions. In the study herein, N. alata plants carrying both the cor15a-IPT gene for dark tolerance and the cor15a-FAD7 gene for cold tolerance, were tested under continuous cold, dark storage. As expected the double transgenic plants showed superior tolerance to prolonged dark, cold storage and the trienoic fatty acid (16:3+18:3) components were higher in the cold-tolerance double transgenic lines and lower in the cold-sensitive wild-type line.

How plants perceive temperature in order to regulate membrane fatty acid desaturation remains an open question (Sakamoto and Murata 2002). However, regulation of fatty acid composition via a molecular genetic approach can be immediately beneficial in commercial agriculture.

In summary, our experiments indicated that deasaturase activity in transgenic plants could be regulated by a cold signal by using the cold inducible cor15a promoter to drive FAD7 expression. The proportion of trienoic fatty acids in leaves of cor15a-FAD7 plants was higher than in wild-type leaves after long-term exposure to cold, and the fatty profile was correlated with a more stable thylakoid ultrastructure and increased survival under prolonged exposure to cold. Further, combining multiple trait genes (such as the isopentenyl transferase (IPT) gene for dark-tolerance and the FAD7 gene for cold-tolerance, under the control of the same cold-inducible promoter can be used to confer selective tolerance to multiple stress conditions.

Definitions

A “transgene” refers to genetic material that is introduced, or is capable of being introduced, into cells of a host animal. Typically, once a “transgene” is introduced into the cells of the host animal, it is maintained, either transiently or permanently, by, e.g., insertion into the host genome. In preferred embodiments of the present invention, a transgene is inserted into the host genome by homologous recombination, thereby replacing the endogenous gene with the transgene. Often, a transgene contains a coding sequence, operably linked to a promoter, that encodes a protein, e.g., a marker protein that allows the detection of the transgene in the cell. “Transgenic” refers to any cell or organism that comprises a transgene.

“Nucleic acid” refers to deoxyribonucleotides or ribonucleotides and polymers thereof in either single- or double-stranded faun. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA and nucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.”

The term “transduction” refers to the introduction of foreign DNA into cells of an organism (in vivo). The term “transfection” refers to the introduction of foreign DNA into cells in culture (in vitro).

The term “vector” commonly refers to a plasmid that can be used to transfer DNA sequences. Different vectors may have properties particularly appropriate for protein expression in the recipient.

“Expression vector” results in expression of inserted DNA when propagated in a suitable host cell such as a plant cell. As used herein, “cassette” may be used to designate a structure into which DNA or a vector may be inserted.

The term “substantial” as used herein refers to being nearly the same, as when expression is substantially the same from a homologous gene. In terms of substantial identity, at least 85% identity is intended.

Use of the terms “an”, “a” and “the” and similar terms used in claiming or describing the invention are intended to be construed as including both the singular and plural, unless clearly otherwise indicated or contraindicated. The terms “including”, “having” and “containing” are to be construed as open-ended in the same manner as the terms “comprising” or “comprises” are commonly accepted as including but not limiting to the explicitly set forth subject matter. The term “comprising” and the like are construed to encompass the phrases “consisting of” and “consisting essentially of”.

The methods and processes described herein may be performed in any suitable order unless otherwise indicated or clearly rendered inoperable by a modification in order.

Limited and narrow interpretation of descriptive language intended to better illustrate the invention is not to be construed as limiting in any way nor to limit the scope of the invention contemplated by the inventors.

The invention, now described generally and in some detail, will be understood more readily by reference to the following examples, which are provided by way of reference and are in no manner intended to limit the scope of what the inventors regard as their invention.

Materials and Methods

Plasmid Construction cor15a-ipt (SEQ ID NO: 10)

Molecular cloning procedures were carried out as described by Sambrook and Russell (2001). The ipt coding sequence used to construct the chimeric cor15a/ipt gene (SEQ ID NO: 10) was derived from the pUC19 vector containing the ipt under the control of the 35S promoter. A 1 kb fragment of ipt-nos was obtained by double digestion of pUC19-35S-ipt-nos with EcoRI and SalI and cloned into the cloning site of the pBluescriptII KS vector. The 5′promoter and leader sequence from the cor15a gene (0.98 kb) was synthesized from genomic DNA of Arabidopsis thaliana by PCR reaction. The cloning sites for XhoI and SalI were added to the ends of the cor15a primers 5′-AGATCTTGTCCGTTGAATTT-3′ (SEQ ID NO: 1) and 5′-AGAGATCTTTAAGATGT-3′(SEQ ID NO: 2) for PCR. The cor15a fragment was subcloned into the pBluescriptII KS-ipt-nos vector by double digestion with XhoI and SalI to generate pBluescriptII KS with ipt under the control of the cor15a promoter. In the final step, the cor15-ipt-nos fragment was inserted into the multicloning site of the pBin19 vector by digesting pBluescriptII KS-cor15a-ipt-nos and pBin19 with KpnI and SmaI and then ligating the blunt ends. Thus, the binary pBin19 vector containing ipt under the control of the cor15a promoter was generated. The binary plasmid was transformed into Agrobacterium tumefaciens strain LBA 4404 by electroporation. The identity and accuracy of the cloned cor15a promoter sequence was confirmed by DNA sequence analysis (W.M. Keck Biotechnology Laboratory, Yale University, New Haven, Conn.).

Plasmid Construction cor15a fad7 (SEQ ID NO: 11)

Molecular cloning procedures were carried out as described by Sambrook and Russell (2001). The promoter sequence from the cor15a gene (0.98 kb) from Arabidopsis thaliana was synthesized from genomic DNA of Arabidopsis by PCR reaction. The initial construction, containing the FAD7 gene sequence from Arabidopsis thaliana, was provided by the Arabidopsis Biological Resources Center (DNA Stock Center) at The Ohio State University. The 1.333 kb FAD7 sequence was released by SacI and XhoI digestion and then subcloned into the same sites of the pBluescript II SK (+/−) vector. The cor15a promoter sequence was cloned via PCR and then subcloned in the SacI site of the pBluescript II SK (+/−) vector, generating the pBluescript II SK-cor15a-FAD7 construct. To establish the final binary vector with the FAD7 gene, we used the pBin19 vector containing the NOS terminator (the pBin19 vector was provided by Dr. Li, University of Connecticut). The vector was cut by SacI and then treated by Klenow fragment to generate a blunt end and then cut by SalI. At the same time the pBluescript II SK-cor15a-FAD7 vector (4.4 kb) was cut by ApaI, treated with Klenow fragment to generate a blunt end, and then cut by SalI to release the cor15a-FAD7 fragment. Finally, this fragment was introduced in pBin19-NOS vector by ligation of one blunt and one sticky end. Thus the binary pBin19 vector containing the FAD7 gene, under the control of the cor15a promoter and ending with the nos terminator, was generated (FIG. 5). The base sequences of the cor15a promoter and FAD7 gene in the plasmid (SEQ ID NO: 11) were confirmed by DNA sequence analysis (W.M. Keck Biotechnology Laboratory, Yale University, New Haven, Conn., USA). The binary plasmid was transformed into Agrobacterium tumefaciens strain LBA 4404 by electroporation.

Transformation and Regeneration of cor15a-ipt Transgenic Petunia & Chrysanthemum

Petunia cv. Marco Polo Odyssey and chrysanthemum cv. Iridon were grown in the 25° C. glasshouse in 3.8 liter pots containing a peat-based substrate (Metro 510, Scotts Co., Marysville, Ohio). Plants were fertilized weekly with 400 mg/l N from a 20N-4.3P-16.6K (Peter's 20-10-20, Scotts Co., Marysville Ohio). Plant shoots were cut at monthly intervals to induce new shoot growth. Leaf and stem tissue from young, newly developed shoots were used as explant tissue for plant transformation as follows.

Young, fully expanded petunia (cv. Marco Polo Odyssey) leaves were sterilized with 0.6% sodium hypochlorite (15-20 min) and then rinsed 5-times with sterile water. Stem from young, soft shoot tips of chrysanthemum plants (cv. Iridon) were washed for 60 sec with 70% ethanol, rinsed 3-times with sterile water, and then sterilized in 5% Clorox for 8 min. before finally rinsing 5-times with sterile water. The bacterial suspension was cultured in LB medium supplemented with 50 mg/L kanamycin and 25 mg/L rifampicin. The suspension was incubated at 25° C. on a rotary shaker (220 rpm) until achieving an optical density of 0.4-0.7 (λ 600 nm). The suspension was then centrifuged and the pellet re-suspended in a fresh liquid MS medium. Leaf explants of petunia or stem segments of chrysanthemum were soaked in the infection medium for 5 min., blotted dry and kept 3 days in the dark at 22-25° C. on plates with MS medium containing 2 mg/L of N⁶-benzyladenine (BA), 0.01 mg/L of NAA for petunia explants or 0.225 mg/L of BA, 2 mg/L of IAA for chrysanthemum explants. After 2-3 days, explants were transferred to the respective selection media containing 50 mg/L of kanamycin (for selection) and 200 mg/L of timentin (to eliminate the Agrobacterium). Explants were transferred to fresh medium every 2-3 weeks, until shoots developed. Excised shoots were then transferred to phytohormone-free MS medium containing 50 mg/L of kanamycin and 100 mg/L of timentin until root induction was evident. Rooted explants were transferred to a peat-based medium (Metro 510, Scotts Co., Marysville, Ohio), and acclimated to the glasshouse environment.

Transformation and Regeneration of Transgenic Tobacco (N. tabacum & N. alata)

Nicotiana tabacum cv. Havana was transformed using an Agrobacterium-mediated transformation protocol (An et al. 1988). Briefly, tobacco leaves were surface-sterilized, cut into discs and co-cultivated with Agrobacterium tumefaciens LBA 4404 bearing the cor15a-FAD7 construct. Following co-cultivation, the explants were transferred to the MS medium supplemented with 0.1 mg/1 of α-naphthaleneacetic acid (NAA), 1 mg/l of 6-benzylaminopurine (BA), 300 mg/l of kanamycin for selection, and 400 mg/l of timentin. Explants were transferred to fresh medium every 2-3 weeks. Shoots developed from the transgenic calli were excised and transferred to hormone-free MS-selection medium (100 mg/l of kanamycin) to induce roots. Plants with roots were transferred in pots and acclimated to the greenhouse. Putative transgenic plants (T₀) were analyzed by PCR for foreign gene integration. T₀ transformants were allowed to self-fertilize in the glasshouse. Seeds of several generations were germinated on MS medium supplemented 100 mg/l kanamycin to determine the segregation patterns of the transgene. Progeny obtained from T₁ plants were analyzed and six homozygous lines (T₂) were used in future cold experiments.

Nicotiana alata cv. Domino was transformed as described above with the following exceptions. Seedling hypocotyls segments (1 cm) were used as explant tissue and the regenerative MS medium was supplemented with 0.1 mg/l of NAA, 3 mg/l of kinetin and 3 mg/l of BA. Kanamycin for selection was used at a concentration 60 mg/l.

Plant DNA Extraction and Polymerase Chain Reaction (PCR Analysis) of cor15a-ipt Plants

Total DNA was isolated from leaf tissue using mini-prep kits (DNeasy Plant Mini Kit, Qiagen Inc., Valencia, Calif., USA) and 250 ng of DNA was subjected to PCR reaction. The primers used to detect the cor15a-ipt locus were as follows: (i) for the full cor15a promoter (0.982 kb fragment), forward primer 5′-AGATCTTGTCCGTTGAA TTT-3′ (SEQ ID NO: 1) and reverse primer 5′-AGAGATCTTTAAGATGT-3′ (SEQ ID NO: 2); and for the 0.523 kb region of ipt gene (ii) forward primer 5-GGTCCAACTTGCACAGGAAAG-3′ (SEQ ID NO: 3) and reverse primer 5′-TAACAAACAACATGGCATATC-3′ (SEQ ID NO: 4). PCR amplification was performed using a thermocycler (GeneAmp PCR System 2700, Applied Biosystems, Inc., Foster City, Calif., USA). Cycling conditions for both genes were 3 min at 94° C. and then 30 cycles of 1 min at 94° C., 1 min at 54° C. and 1 min 30 sec at 72° C., followed by extension at 72° C. for 5 min. The reactions included 200-250 ng of DNA template, 0.2 mM of dNTPs, 0.5 μM of each primer, REDTaq PCR Buffer and 1.5 u of REDTaq DNA polymerase (Sigma, Saint Louis, Mo., USA). Finally, a 20 μl aliquot of PCR product was observed under UV after electrophoresis on a 1% agarose gel with ethidium bromide. A 1-kb DNA molecular marker (Gibco BRL) was used as a reference to determine DNA fragment size.

Plant DNA Extraction and Polymerase Chain Reaction (PCR Analysis) of cor15a-fad7 Plants

Total DNA was isolated from leaf tissue of primary putative transgenic plants of Nicotiana tabacum, as well as T₁ and T₂ generative plants, by DNeasy Plant Mini Kits (Qiagen Inc., Valencia, Calif., USA) and 200 ng of DNA was subjected to PCR reaction. The primer pairs used for DNA amplification were 5′-AGATCTTGTCCGTTGAATTT-3′ (SEQ ID NO: 1); and 5′-AGAGATCTTTAAGA TGT-3′ (SEQ ID NO: 2) to release the 0.982 kb fragment of the cor15a promoter and 5′-GGTATACGACCTCTCCCC-3′ (SEQ ID NO: 5); 5′-GGTCCAGACTTATCA GGC-3′ (SEQ ID NO: 6) to release the 1.176 kb fragment of the FAD7 gene. PCR amplification was performed using a thermocycler (GeneAmp PCR System 2700, Applied Biosystems, Inc., Foster City, Calif., USA). Cycling conditions for cor15a promoter were: 3 min at 94° C.; 30 cycles of 1 min at 94° C., 1 min at 54° C. and 1 min 30 sec at 72° C.; extension at 72° C. for 5 min. Cycling conditions for 1.25 kb fragment of FAD7 gene were: 3 min at 94° C.; 30 cycles of 1 min at 94° C., 1 min at 57° C. and 1 min 30 sec at 72° C.; extension at 72° C. for 5 min. The reactions involved 200 ng of DNA template, 0.2 mM of dNTPs, 0.5 mM of each primer, 1× REDTaq PCR Buffer and 1 u of REDTaq DNA polymerase (Sigma, Saint Louis, Mich., USA) in a final reaction volume of 20 μl. PCR products were observed under UV after electrophoresis on a 1% agarose gel with ethidium bromide. A 1-kb DNA molecular marker was used as a reference (Gibco BRL, Carlsbad, Calif., USA). Transgenic plants of Nicotiana alata containing the cor15a-FAD7 construction were confirmed using the same procedure. Putative cor15a-IPT transgenic lines of Nicotiana alata were confirmed by PCR analysis according the protocol reported by Khodakovskaya et al. (2005). Cor15a-IPT and cor15a-FAD7 Nicotiana alata parent lines were crossed to produce double transgenic (cor15a-IPT×cor15a-FAD7) T₁ plants. Putative double transgenic (cor15a-IPT×cor15a-FAD7) and plants of T₁ generation plants were confirmed by PCR analysis.

Southern Hybridization

Total genomic DNA was isolated from transgenic plants using DNeasy Plant Maxi Kits (Qiagen Inc., Valencia, Calif., USA) in accordance with the recommended protocol. Total genomic DNA from putative transgenic and non-transformed control plants (10 mg samples) was digested at 37° C. overnight by double restriction with enzymes HindIII and EcoRI and cor15a-ipt fragment was released. Digested DNA from each line was separated through a 1% agarose gel prepared in TAE buffer, pH 8.5 (Sambrook and Russell, 2001) and fragments were transferred from agarose gel to a nylon membrane (Amersham, Chalfont St Giles, UK) and cross-linked to the membrane under UV irradiation. The ipt gene probe (a 0.523 kb fragment of the ipt gene) was prepared with a PCR DIG Probe synthesis kit (Roche Molecular Biochemicals, Indianapolis, Ind.) in accordance with the recommended protocol. The DNA fixed on membranes was prehybridized using a prehybridization solution at 68° C. for 3 h, and then hybridized with the probe at 68° C. overnight, and finally triple-washed with the post-hybridization solution at 65° C. in a hybridization oven (HB-2D, Techne Ltd., Duxford-Cambridge, UK). Solutions for sample hybridization, and pre- and post-hybridization, and the buffers for the following steps were prepared as previously reported by Mercier (1998). Membranes were washed for 5 min. in 50 ml of maleate buffer (0.1 M maleic acid, 3.0 M of NaCl, pH 8.0) at room temperature and then incubated for 1 hour in 50 ml of blocking solution (maleate buffer plus 0.5% blocking reagent (Roche Molecular Biochemicals, Indianapolis, Ind.). Membranes were then incubated for 30 min. in 20 ml of blocking solution with anti-dioxigenin-AP, Fab fragments (Roche Molecular Biochemicals, Indianapolis, Ind.) diluted to 1:10,000 and then washed 4-times for 10 min. in 50 ml of the maleate buffer. As a final step, membranes were equilibrated for 5 min. in 50 ml of substrate buffer (100 mM of Tris-HCl; 100 mM of NaCl; 5 mM of MgCl₂, pH 9.5) and then incubated at 37° C. for 10 min in 2 ml (sandwiched between two translucent plastic pages) of substrate buffer plus chemiluminescent substrate at a 1:100 dilution (CSPD, Roche Molecular Biochemicals, Indianapolis, Ind.). Membranes were exposed to autoradiographic film (Kodak X-Omart AR) for 4 hours. X-ray films were developed with an automatic film processor.

Southern Hybridization with cor15a-fad7 Plants

Three kanamycin-resistant transgenic lines of the Nicotiana tabacum (T₂ generation) were analyzed by Southern blot. Total genomic DNA was isolated from transgenic plants using DNeasy Plant Maxi Kits (Qiagen Inc., Valencia, Calif., USA) in accordance with the recommended protocol. Total genomic DNA from putative transgenic and non-transformed control plants (10 μg samples) was digested at 37° C. overnight by double restriction with enzymes HindIII and EcoRI and the cor15a-FAD7 fragment was released.

Digested DNA from each line was separated through a 1% agarose gel prepared in TAE buffer pH 8.5 (Sambrook and Russell 2001) and fragments were transferred from agarose gel to a nylon membrane (Amersham, Chalfont St Giles, UK) and cross-linked to the membrane under UV irradiation. The FAD7 gene probe (for 1.25 kb fragment) was prepared with a PCR DIG Probe synthesis kit (Roche Molecular Biochemicals, Indianapolis, Ind.) in accordance with the recommended protocol. The DNA fixed on membranes was prehybridized using a prehybridization solution at 68° C. for 3 h, and then hybridized with the probe at 68° C. overnight, and finally triple-washed with the post-hybridization solution at 65° C. in a hybridization oven (HB-2D, Techne Ltd., Duxford-Cambridge, UK). Solutions for sample hybridization, and pre- and post-hybridization, and the buffers for the following steps were prepared as previously reported by Mercier (1998). Membranes were washed for 5 min. in 50 ml of maleate buffer (0.1 M maleic acid, 3.0 M of NaCl, pH 8.0) at room temperature and then incubated for 1 hour in 50 ml of blocking solution consisting of maleate buffer plus 0.5% blocking reagent (Roche Molecular Biochemicals, Indianapolis, Ind.). Membranes were then incubated for 30 min. in 20 ml of blocking solution with anti-dioxigenin-AP, Fab fragments (Roche Molecular Biochemicals, Indianapolis, Ind.) diluted to 1:10,000 and then washed 4-times for 10 min. in 50 ml of the maleate buffer. As a final step, membranes were equilibrated for 5 min. in 50 ml of substrate buffer (100 mM of Tris-HCl; 100 mM of NaCl; 5 mM of MgCl₂, pH 9.5) and then incubated at 37° C. for 10 min in 2 ml (sandwiched between two translucent plastic pages) of substrate buffer plus chemiluminescent substrate at a 1:100 dilution (CSPD, Roche Molecular Biochemicals, Indianapolis, Ind.). Membranes were exposed to autoradiographic film (Kodak X-Omart AR) for 4 hours. X-ray films were developed with an automatic film processor.

Analysis of ipt Expression in Leaves of Petunia and Chrysanthemum

Using wild type and two cor15a-ipt transgenic lines from both petunia and chrysanthemum, total RNA was isolated with TRI reagent (Molecular Research Center, Inc., Cincinnati, Ohio, USA) from samples that were frozen with liquid nitrogen and then ground in a mortar. For RT-PCR analysis, DNase treatment (DNA-free™, Ambion, Inc.) was used to eliminate DNA contamination from RNA samples, and then first-strand cDNA was synthesized from 1 μg of total RNA using First Strand Synthesis Kit RETROscript™ (Ambion Inc. Austin, Tex., USA) following the manufacturer recommended protocol. For PCR, 0.5 μL of RT-mix was used in a final volume of 25 μL. PCR reaction for the ipt gene fragment was carried out as described above. PCR reaction products along with RT-mix and primers to 18S RNA were used as internal standards (QuantumRNA™ 18S Internal Standards, Ambion Inc.). PCR products (10 μL) were run on a 1% agarose gel.

Analysis of FAD7 Gene Expression in Leaves of Tobacco

Total RNA was isolated from plants of wild type and transgenic tobacco line N2 (a line which was found to be cold tolerant in preliminary trials) by grinding previously frozen samples in a mortar with TRI reagent (Molecular Research Center, Inc., Cincinnati, Ohio, USA). For RT-PCR analysis, DNA contamination was removed from RNA samples by DNase treatment (DNA-free™, Ambion, Inc., Austin, Tex., USA), and then first-strand cDNA was synthesized from 1 μg of total RNA using First Strand Synthesis Kit RETROscript™ (Ambion Inc., Austin, Tex., USA) following the recommended protocol. For PCR, a 0.5 μL aliquot of RT-mix was used in a final volume of 25 μL. PCR reaction for the 1.176 kb fragment of the FAD7 gene was conducted as previously described. PCR reaction products along with RT-mix and primers to 18S RNA were used as internal standards (QuantumRNA™ 18S Internal Standards from Ambion Inc., Austin, Tex., USA). PCR products (10 μL) were run on a 1% agarose gel.

Senescence of Excised Leaves and Shoots

To determine transgenic plant tolerance to prolonged dark storage, excised leaves from petunia and chrysanthemum were surface sterilized with 0.6% sodium hypochlorite for 60 sec, rinsed 5 times and then placed on moist filter paper in a 10 cm Petri dish. Leaves were selected from individual transgenic lines and non-transformed wild type plants. Each Petri dish contained two excised leaves from both an individual transgenic plant line and a non-transformed wild type plant. For petunia, five transgenic lines were tested. For chrysanthemum, two transgenic lines were tested. Identical plates were assembled for each transgenic line of petunia and chrysanthemum. The plates were either exposed to (1) a cold induction period (3 d at 4° C. in the dark) followed by continuous dark storage at 25° C., or (2) continuous dark storage at 25° C. without a prior cold induction treatment. Plates in dark storage were checked daily over a 28-day period for evidence of leaf senescence. Chlorophyll concentration was assayed prior to the start of each experiment and after significant loss of chlorophyll was detected in the non-transformed wild type tissue. Each treatment combination was replicated in triplicate and the experiment was repeated three times. In separate experiments, whole shoots were excised from both wild type plants and individual transgenic lines of petunia and chrysanthemum. Shoots from individual plants were bundled in groups of five and wrapped in a moist paper towel. Bundles from each transgenic line and the wild type were enclosed in a plastic bag and subjected to the same treatments and experimental protocol as previously described.

Quantification of Chlorophyll in Wild-Type and Transgenic Plants

The specific chlorophyll concentration was determined as follows. The youngest, fully expanded leaf was sampled from individual plants in each sample group (the wild type and each transgenic line tested). Leaves were blotted dry, and a 100 mg sample leaf was placed in a 1.5-mL sample tube. The samples were resuspended in 80% acetone, ground with a disposable pestle, and incubated in the dark for 30 min. Total chlorophyll (Chl μg mL⁻¹) was determined using absorbance at 645 and 663 nm, according to the equation: 20.2 A₆₄₅+8.02 A₆₆₃ (Chory et al. 1994). The average of chlorophyll content was determined from 10 independent plants from each sample group both before and after cold.

Cultivation and Cold-Resistance Analysis of Transgenic Plants

Transgenic lines, which exhibited ‘normal’ morphology (no differences in growth or flowering from the wild type) and were found to be most cold tolerant in preliminary trials, were selected for use in this and subsequent experiments. Further the selected transgenic lines conformed to Mendelian principles of inheritance for a single genetic locus. Young T₂ seedlings from each of six homozygous transgenic lines and the wild type were arranged into identical groups of 12 (on 288 plug trays) when the seedlings reached the 4-true leaf stage. Seedlings were then exposed to a cold-induction signal (4° C. for 4 days) before returning to growth conditions (25° C.) for one week. Plants were then transferred to cold-stress conditions at 0.5, 2 or 3.5° C. Seedlings under cold-stress were exposed to light at 10 μM·m⁻²·s⁻¹ and watering as needed to prevent desiccation. After 44 days of exposure to cold the number of dead and the number of live seedlings from each treatment group were counted, and the percentage of surviving seedlings was calculated. Plants were considered dead when the entire shoot collapsed and the shoot apex appeared necrotic. Plants were considered alive, regardless of turgor status and leaf color, as long as the shoot apex was not necrotic and the shoot has not collapsed. The experiment was replicated in triplicate overtime. Data were analyzed as a two-way analysis of variance with genetic line and storage temperature as the independent variables.

Morphological Analysis of Transgenic cor15a-ipt Plants

The effect of the transgene on growth and development of chrysanthemums was determined in growth chamber studies. Thirty shoots from each transgenic cor15a-ipt chrysanthemum plant lines #9 and #12, and from the wild type cultivar ‘Iridon’ were excised and rooted in deep 606-cell packs (Kord Products, Bramalea, Ontario, Canada) containing a Metro 510 (Scotts Co., Marysville, Ohio) peat-lite medium. After shoots were well rooted (3 weeks), the rooted cuttings were transferred to the growth chamber (EGC model S10, EGC, Chargrin Falls, Ohio) at 25° C. day (16 hours at 300 μmol·m⁻²·s⁻¹) and 20° C. night. Plants were allowed to acclimate to the growth chamber conditions for 2-weeks and then 10 plants from each line were exposed to a cold induction period (3 days at 4° C.) and then returned to the growth chamber while 10 plants from each line remained in the growth chamber without exposure to a cold induction period. In the growth chamber, plants were watered as needed and fertilized once per week with N at 5.3 mmol (75 mg L⁻¹) from 20N-4.3P-16.6K (Peter's 20-10-20, Scotts Co., Marysville Ohio). After six weeks in the growth chamber five plants from each treatment were harvested and the following data recorded: shoot fresh weight (g), number of lateral shoots, lateral shoot length (cm), number of secondary shoots on each lateral, leaf area (cm²) on upper-most lateral shoot, number of nodes on the upper-most lateral shoot, and total number of lateral shoots on the main stem. These parameters were used to calculate the average internode length and the average area per leaf on the upper-most lateral shoot. These data were used to determine difference in vegetative growth habit between transgenic and wild type plants with or without exposure to a cold-induction period. In a separate study, the remaining 10 rooted chrysanthemum cuttings from each line were exposed to short-day conditions to induce flowering and the number of flower buds on each plant were recorded at anthesis.

Plants were arranged in a randomized complete block design with 10 replicated blocks. Statistical effects were determined using a two-way analysis of variance with genetic line and cold-treatment as the main effects.

Cytokinin Analysis

Cytokinins were extracted and purified according to the method of Dobrev and Kaminek (2002). Freeze-dried samples were homogenized with mortar and pestle in liquid nitrogen and extracted overnight with 10 ml methanol/water/formic acid (15/4/1, v/v/v, pH˜2.5, −20° C.). To each sample, 50 ρmol of each of twelve deuterium labelled standards ([²H₅]Z, [²H₅]ZR, [²H₅]Z7G, [²H₅]Z9G, [²H₅]ZOG, [²H₅]ZROG, [²H₆]iP, [²H₆]iPA, [²H₆]iP7G, [²H₆]iP9G, [²H₅]DHZ, [²H₅]DHZR; products of Apex Organics, Honiton, UK) were added. The extract was passed through 2 ml Si—C₁₈ columns (SepPak Plus, Waters, USA) to remove interfering lipophilic substances. After organic solvent evaporation in vacuo, the aqueous residue was applied to an Oasis MCX mixed mode (cation exchange and reverse-phase) column (150 mg, Waters, USA). Adsorbed cytokinins were eluted stepwise with 5 ml of 0.35 M ammonium in water (cytokinin nucleotides) and 0.35 M ammonium in 60% methanol (v/v) (cytokinin bases, ribosides and glucosides). The eluted fractions were evaporated in vacuo. Nucleotide samples were dephosphorylated with acid phosphatase (0.6 U per sample) for 1 h at 37° C. LC-MS analysis was performed using a Rheos 2000 HPLC gradient pump (Flux Instruments, Basel, Switzerland) and HIS PAL autosampler (CTC Analytics, Zwingen, Switzerland) coupled to an Ion Trap Mass Spectrometer Finnigan MAT LCQ-MSⁿ equipped with an electrospray interface. Samples dissolved in 10% (v/v) acetonitrile (10 μl) were injected on an C₁₈ column (Aqua 125A, 2 mm/250 mm/5 μm) and eluted with a linear gradient of B from 10% to 50% in 26 min (mobile phase: water (A), acetonitrile (B) and 0.001% (v/v) acetic acid in water (C) at a flow rate 0.2 ml/min. Under these chromatographic conditions all analyzed cytokinins were separated. Detection and quantification were carried out using a Finnigan LCQ operated in the positive ion, full-scan MS/MS mode using a multilevel calibration graph with deuterated cytokinins as internal standards. The levels of 19 different cytokinin derivatives were measured. The detection limit was calculated for each compound as 3.3σ/S, where σ is the standard deviation of the response and S the slope of the calibration curve. For each treatment, samples were collected from each of three independent plants and each sample was injected at least twice.

Fatty Acid Analysis

The youngest fully expanded leaves were used for the fatty acid extraction. Leaf material (200 mg FW) from each line studied was lyophillized using a freeze dry system/freezone 4.5 (Labconco Inc., Kansas City, Mo., USA). The extracts from the dried material were then prepared as described in Wang et al. (1996). Briefly, the lipids in the freeze-dried tissue were acidified with 1N H₂SO₄ and the fatty acids were methylated with heating at 80° C. for 90 min. A solution containg 0.9% NaCl and 200 μmol hexane was added to the sample and then vortexed. After centrifuging for 5 min at 250×g, 3 μl of the sample was subjected to GC-MS analysis. Fatty acids were separated and identified with a HP GG-MS (HP 6890 GC and HP 5973 MS) (Hewlett Packard Co., Palo Alto, Calif., USA). A 60-m HP-5MS capillary column with an ID of 0.25 mm was used. The GC was programmed to begin at 170° C. for 10 min, followed by a 10 min ramp until 220° C., at a flow rate of 1 ml/min. Leaf tissue was sampled from both wild type and transgenic (cor15a-FAD7 line N2) seedlings, before and after exposure to 4° C. for 40 days. Samples were collected from three independent plants from each test group. A standard T-test was used to compared changes in fatty acid composition before and after cold-induction and between wild type and transgenic plants

Membrane Imaging Using Electron Microscopy

Both the transgenic line N2 and wild-type tobacco were used for microscopic analysis. Leaf samples were collected from five plants from each genetic source before cold treatment and then again after cold treatment. Leaves that appeared representative of the plant response in each treatment group were selected. Whole leaves were pinned onto Silgard-coated plastic petri dishes and overlaid with a fixing solution containing 2% paraformaldehyde, 2.5% glutaraldehyde, 1.5 mM calcium chloride (CaCl₂) and 1.5 mM (MgCl₂) IN 0.05 M PIPES buffer, pH 6.9. Small (1 mm×2 mm) pieces were then cut with a razor blade from the apical leaf tips and pinned in place to keep them submerged. Dishes were covered and fixation proceeded for 5.5 hrs at room temperature. Thereafter, leaf pieces were washed 3 times for 20 min each in 0.05 PIPES buffer containing 1.5 mM CaCl₂ and 1.5 MgCl₂ and placed at 4° C. in the same solution overnight. Samples were washed one more time in the buffer rinse and then briefly postfixed at room temperature for 20 min in 1% osmium tetroxide, 0.8% potassium ferricyanide, 1.5 mM CaCl₂ and 1.5 mM MgCl₂ in 0.05 M PIPES buffer, pH 6.9, after which time Kodak Photo-flo was added (3.5% v/v) as a surfactant to reduce surface tension. After several minutes, pieces were unpinned from the petri dishes and transferred to small shell vials containing fresh fixative without Photo-flo. Post-fixation continued for an additional 2.25 hrs. After fixing, tissues were returned to 4° C. by rinsing in cold distilled water, 3 times for 20 min each, and dehydrated in a ascending ethanol series from 10% to 70% ethanol (EtOH), in 10% increments for 20 min each. Tissues were then stained in 1% uranyl acetate in 70% EtOH for 1.5 hr at 4° C., followed by two 5 min rinses in 70% EtOH, with the temperature brought back to room temperature during the second rinse. Dehydration was continued by washing tissues once in 85% and 95% EtOH and twice in 100% EtOH, 15 to 20 min per step. Finally, two washes in propylene oxide for 10 min each preceded the embedment of material into Spurr's resin. Thin sections were cut from the embedded samples using an ultramicrotome equipped with a diamond knife. Sections were mounted on copper grids (200 mesh), stained with lead citrate, and examined under a transmission electron microscope (Philips EM 300, Eindhoven, The Netherlands) at the University of Connecticut Electron Microscope Laboratory. TEM images were viewed for ten thin sections prepared from each leaf sampled. Twelve chloroplasts of each section were observed and representative images for each treatment group were recorded.

Assessin Survival of Double Transgenic Nicotiana alata Seedlings or Tolerance to Cold, Dark Conditions

Double transgenic Nicotiana alata seedlings were generated, by crossing cor15a-FAD7and cor15a-IPT parent lines. Seeds (T₁ generation) were germinated in Petri dishes on MS media with 60 mg/l kanamycin. Kanamycin resistant seedlings were transferred to a soil-less medium in 288 plug trays and acclimated to the greenhouse. A small number of plants from each genetic cross were exposed to cold-inductive temperatures and RT-PCR was used to confirm expression of both the IPT and FAD7 genes. Double transgenic lines #33 and #41 were found to express both genes in response to a cold signal and these lines were used to evaluate cold, dark tolerance as follows. Once seedlings reached the 4-true leaf stage and the roots were well established in the medium, 10 seedlings from each double transgenic line #33 and #41 and 10 wild type plants, were placed into a parallel rows on a fresh plug sheet. The plug sheet arrangement was repeated three times. Plants were then cold-induced at 4° C. for 3-days, returned to the greenhouse at 25° C. for 5 days and then placed in a dark, temperature controlled cooler at 2° C. for 50 days. Plants were checked regularly, watered as needed and assessed for visible signs of injury. At the end of the 50-day test period, plant survival was assessed as previously described. The entire experiment was repeated with fresh seedlings. Survival response data were analyzed as a one-way analysis of variance. Finally, the fatty acid profile from double transgenic N. alata lines and wild type plants was analyzed as previously described.

### EXAMPLE

### Gene Construction and Plant Transformation (cor15a-ipt)

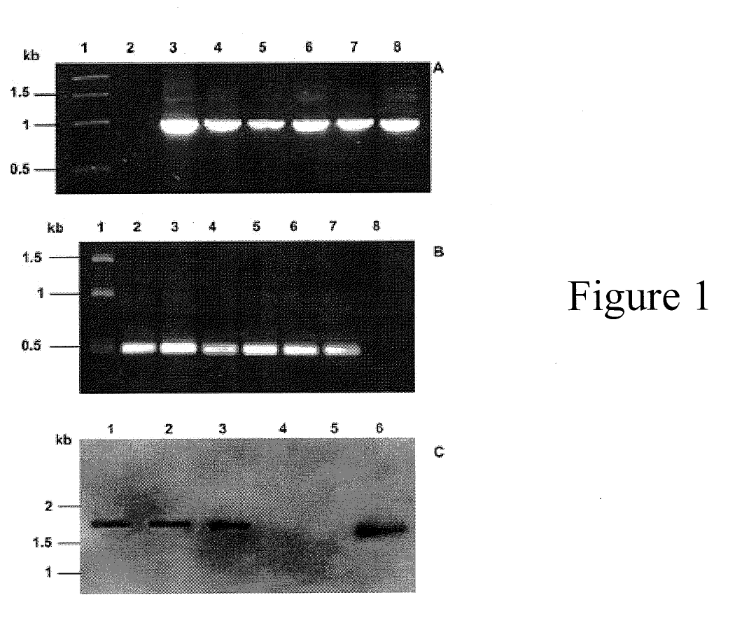

Transformation of petunia and chrysanthemum with the cor15a gene promoter-ipt gene (cor15a-ipt) construct resulted in more than 30 kanamycin-resistant putative transformants for each species. PCR and Southern hybridization analysis confirmed recombinant DNA integration into the genome of individual putative-transgenic petunia and chrysanthemum lines (FIG. 1). PCR amplification of both plasmid DNA and the genomic DNA from chrysanthemum lines produced the expected 0.982 kb fragment of the cor15a promoter (FIG. 1A) and the 0.523 kb fragment of the ipt gene (FIG. 1B). No amplification of DNA was detected in non-transgenic plants. Southern blot analysis of petunia genomic DNA revealed the integration of the ipt gene into the genome of several primary transformants, while no signal was detected in control plants (FIG. 1C). Transgenic plants of petunia were also confimied by PCR reaction, and PCR positive lines of petunia and chrysanthemum were used for all subsequent experiments.

### EXAMPLE

### Gene Construction and Plant Transformation (cor15a-fad7 SEQ ID NO:11)

Agrobacterium transformation of tobacco with the cor15a-fad7 construct resulted in more than 20 kanamycin-resistant putative transformants. PCR and Southern hybridization analysis confirmed recombinant DNA integration into the genome of individual putative-transgenic plants (FIG. 6) and plants of generations T₁ and T₂. PCR amplification of both plasmid DNA and the genomic DNA from tobacco lines produced the expected 0.982 kb fragment of the cor15a promoter (FIG. 6A) and the 1.176 kb fragment of the FAD7 gene (FIG. 6B). No amplification of DNA was detected in non-transgenic plants. Southern blot analysis of genomic DNA revealed the integration of different copy numbers of FAD7 gene into the genome of several T₂ lines; lines1 and 3 contained a single T-DNA copy, line 2 carried two copies. No signal was detected in control plants (FIG. 6C).

### EXAMPLE

### Molecular Analysis of Transgenic Plants Expressing cor15a-ipt (SEQ ID NO: 10)

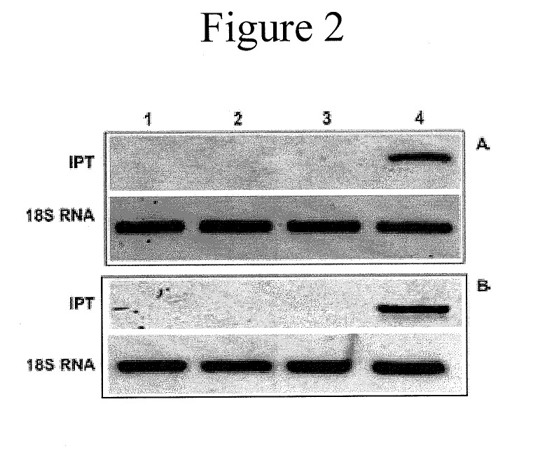

Reverse transcription-PCR (RT-PCR) analysis was used to confirm ipt expression in transgenic lines in response to cold-induction signal. Total RNAs were extracted from the leaves of wild type and selected transgenic lines of chrysanthemum (lines 9 and 12) and petunia (lines 7 and 9) that were grown under normal conditions or first exposed to a 3-day cold-induction (4° C.) treatment. RT-PCR analysis showed that the 0.523 kb ipt DNA fragment was amplified in both cor15a-ipt chrysanthemum (line 9) and cor15a-ipt petunia (line 7) exposed to the 4° C. treatment but not in the same lines grown at the 25° C. and not exposed to the cold-induction treatment (FIG. 2). Similar results were obtained with line 9 of petunia and 12 line of chrysanthemum (data not shown). Wild type plants showed no evidence of ipt gene expression regardless of temperature treatment. These data demonstrate that ipt expression in cor15a-ipt plants could be up regulated with a cold-induction signal but remained suppressed at normal growing temperatures.

### EXAMPLE

### Molecular Analysis of Transgenic Plants Expressing FAD7 Under the Control of a Cold-Inducible Promoter

Reverse transcription-PCR (RT-PCR) analysis wild type and selected transgenic tobacco lines revealed a strong FAD7 gene transcription signal in cor15a-FAD7 tobacco exposed to the 4° C. cold-induction treatment but the transcription signal was very weak in cor15a-FAD7 tobacco plants (line N2) that were not exposed to the cold-induction treatment (FIG. 7). Wild type plants showed no evidence of FAD7 gene expression regardless of temperature treatment. These data demonstrate that FAD7 expression in cor15a-FAD7 plants could be dramatically up regulated via a cold-induction signal.

### EXAMPLE

### Delayed Leaf Senescence in cor15a-ipt Plants

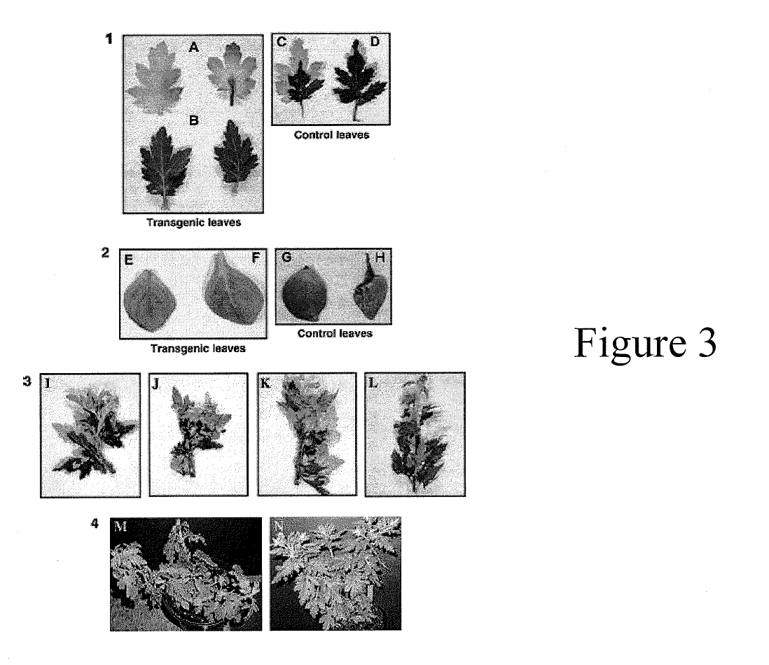

The leaf senescence response of chrysanthemum and petunia under long-term dark storage conditions differed markedly between cor15a-ipt and wild type plants (FIG. 3). Overall, leaves from cold-induced cor15a-ipt plants remained green and healthy in prolonged dark storage while leaves from non-induced cor15a-ipt plants and from wild type plants, regardless of cold-induction treatments, did not. Similar results were observed with excised leaves of both chrysanthemum and petunia, and excised shoots and whole intact plants of chrysanthemum. For example, excised leaves of both wild type chrysanthemum and wild type petunia showed a dramatic loss of chlorophyll and advanced tissue senescence after 28 days in continuous darkness at 25° C. A pre-treatment of cold-induction temperatures had little effect on the course of tissue senescence under these conditions. However, when excised leaves of cor15a-ipt chrysanthemum and cor15a-ipt petunia were exposed to a cold-induction treatment (4° C. for 3 days) and then stored in the dark for 28 days, the tissue showed little or no visible symptoms of chlorophyll loss or tissue senescence. Leaves of cor15a-ipt chrysanthemum and cor15a-ipt petunia that were not exposed to a cold-induction treatment prior to dark storage developed symptoms of chlorophyll loss or tissue senescence that approached those observed in wild type leaves. With both excised shoots of cor15a-ipt chrysanthemum and whole intact cor15a-ipt chrysanthemum plants, a cold-induction treatment was required to produce the delayed onset of leaf senescence response under prolonged dark storage conditions.

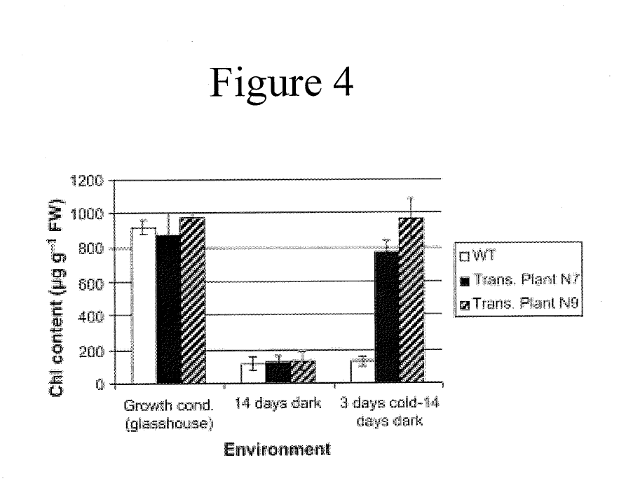

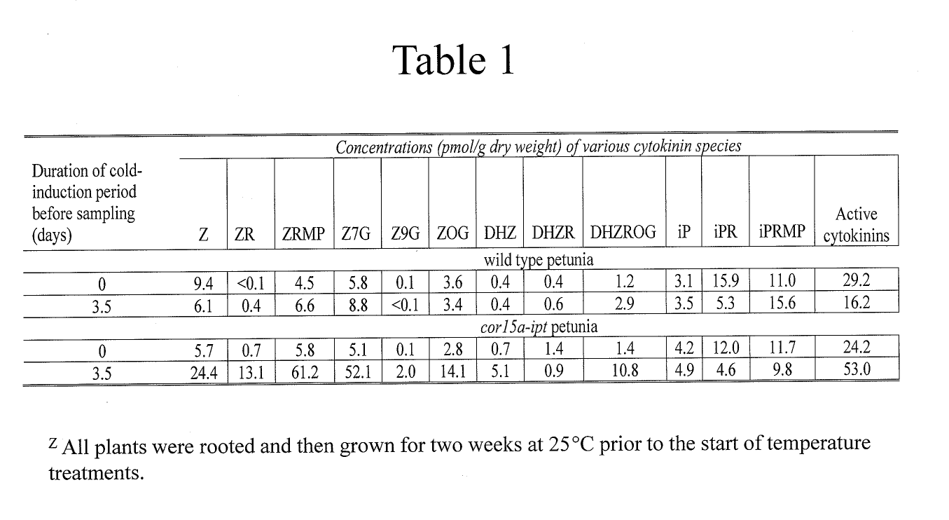

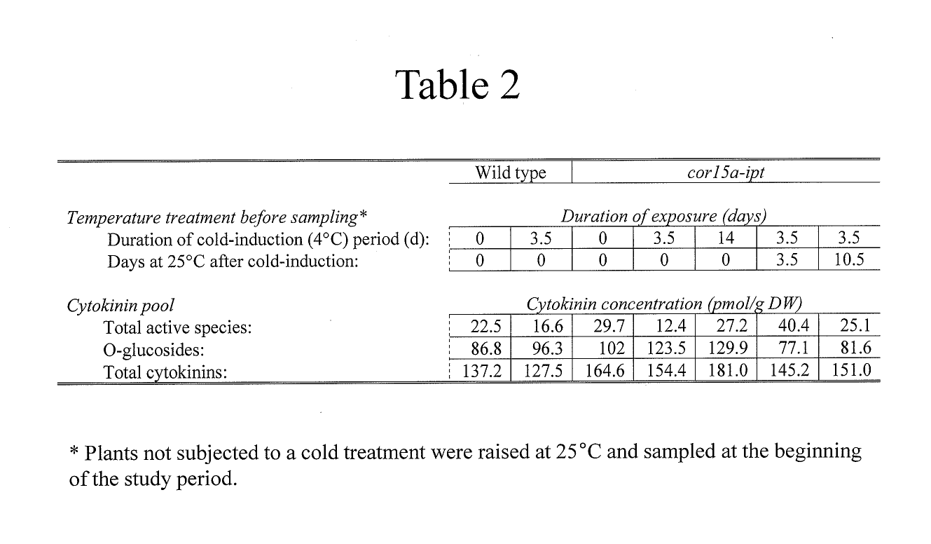

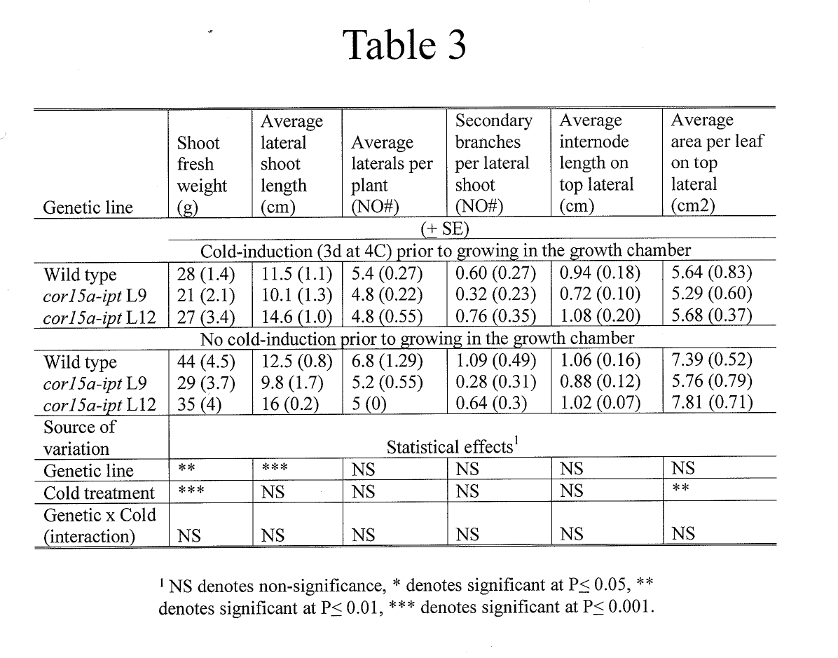

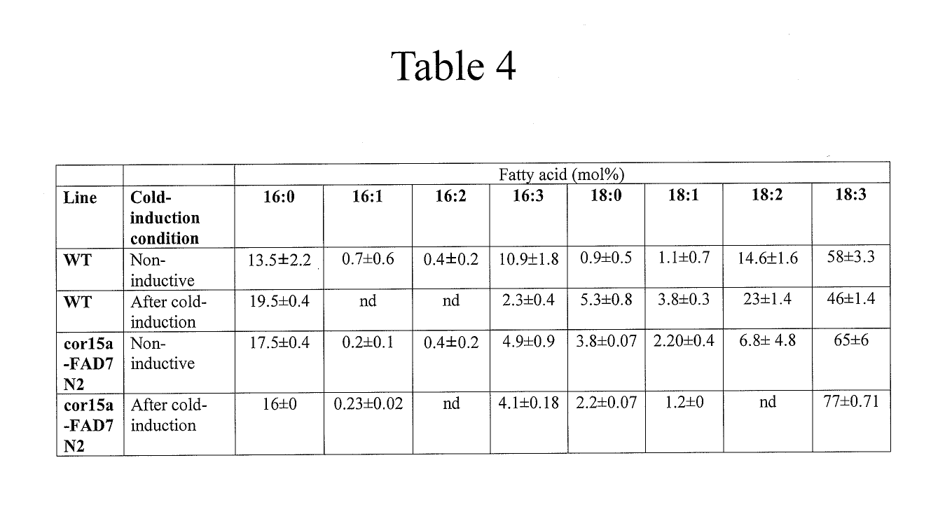

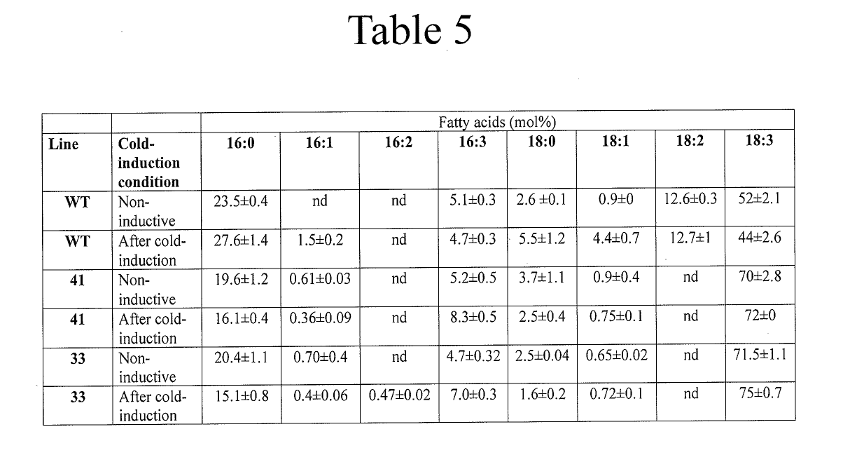

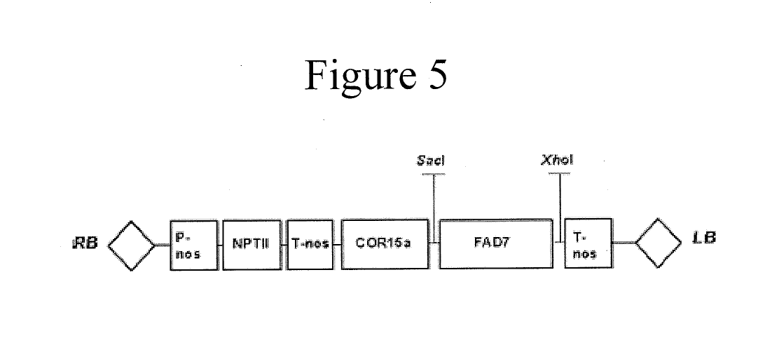

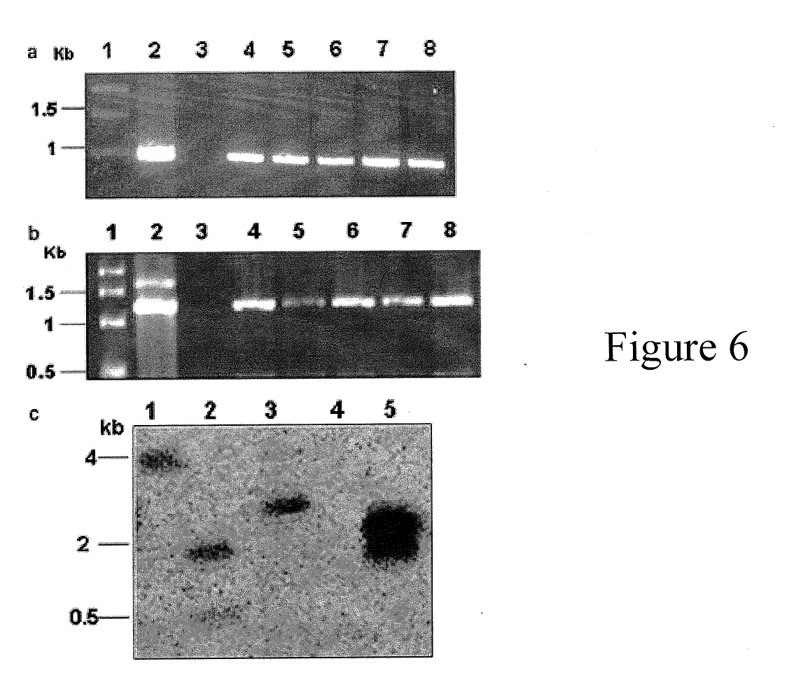

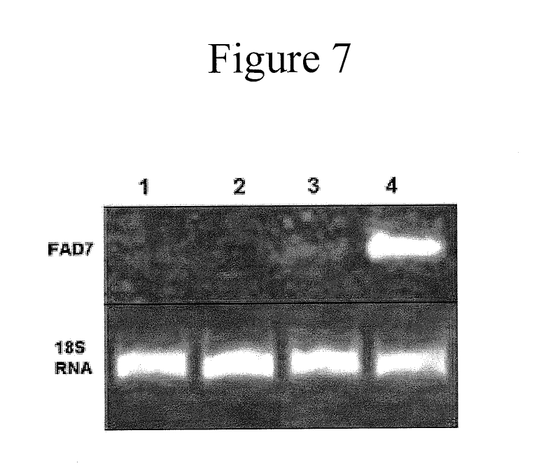

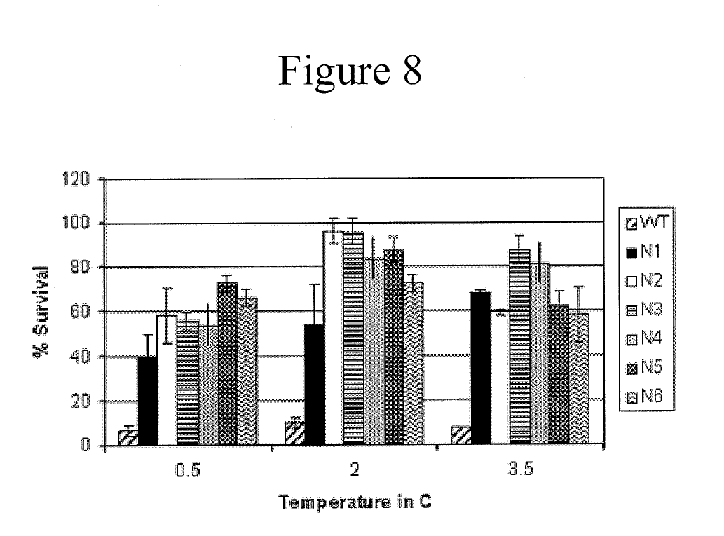

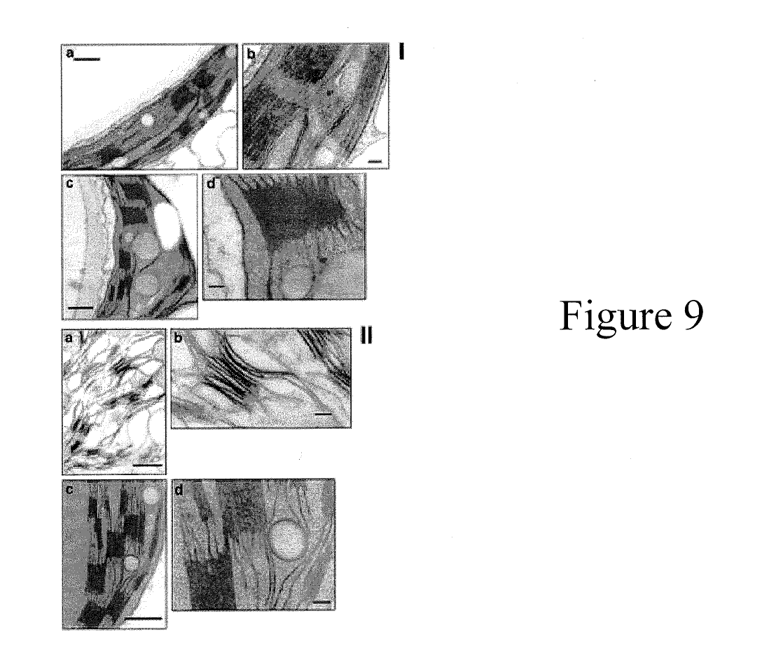

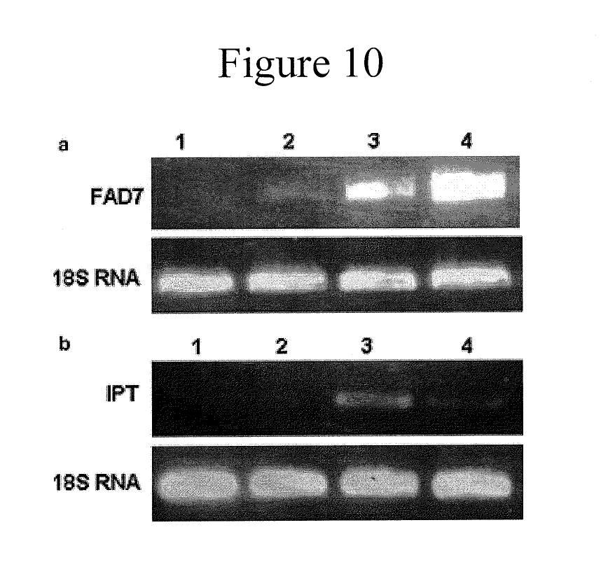

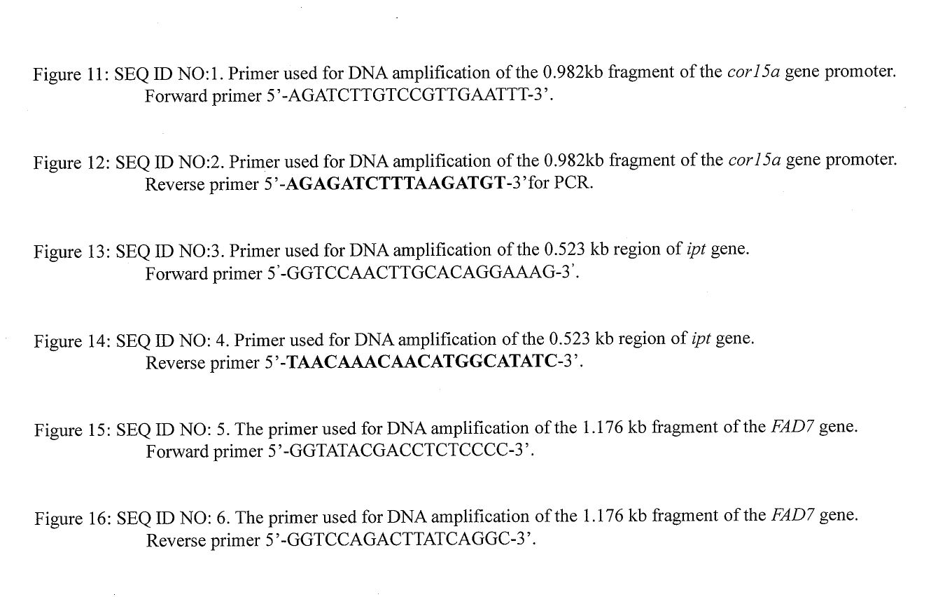

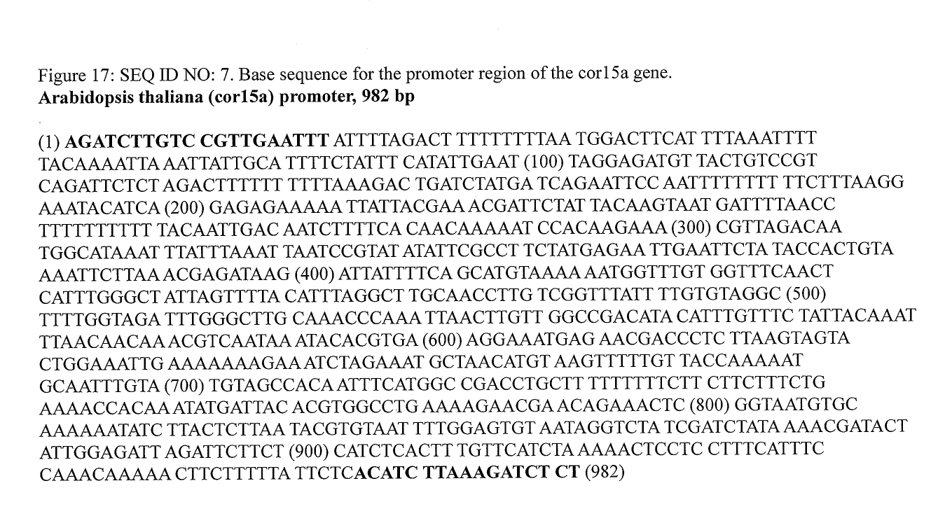

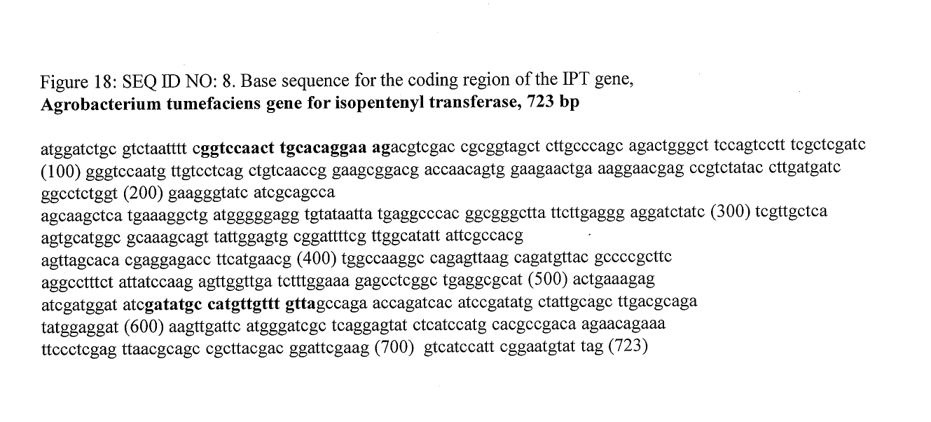

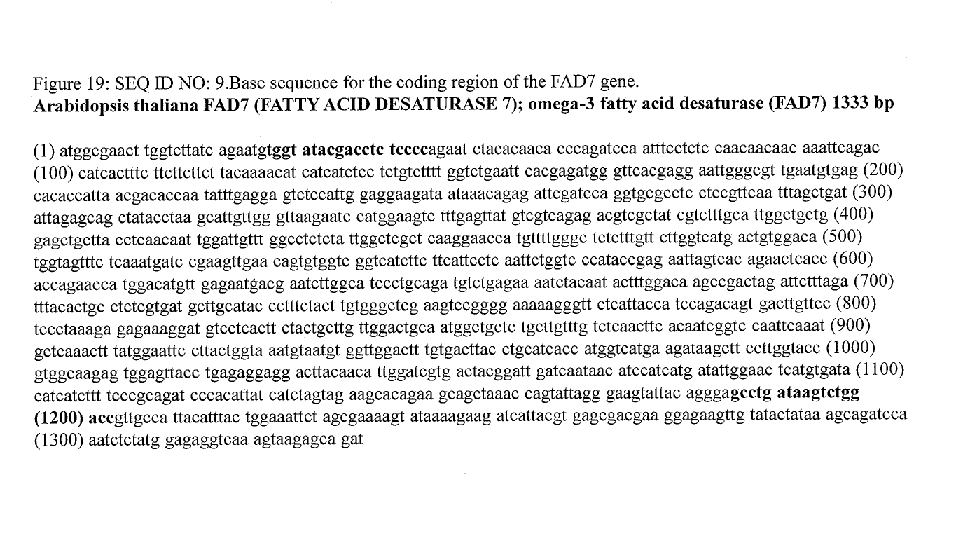

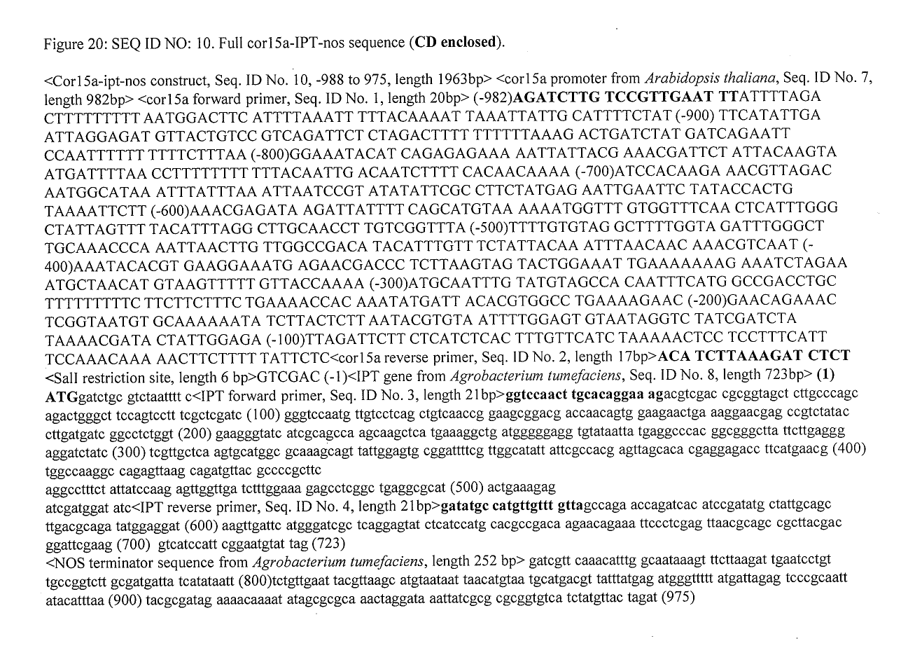

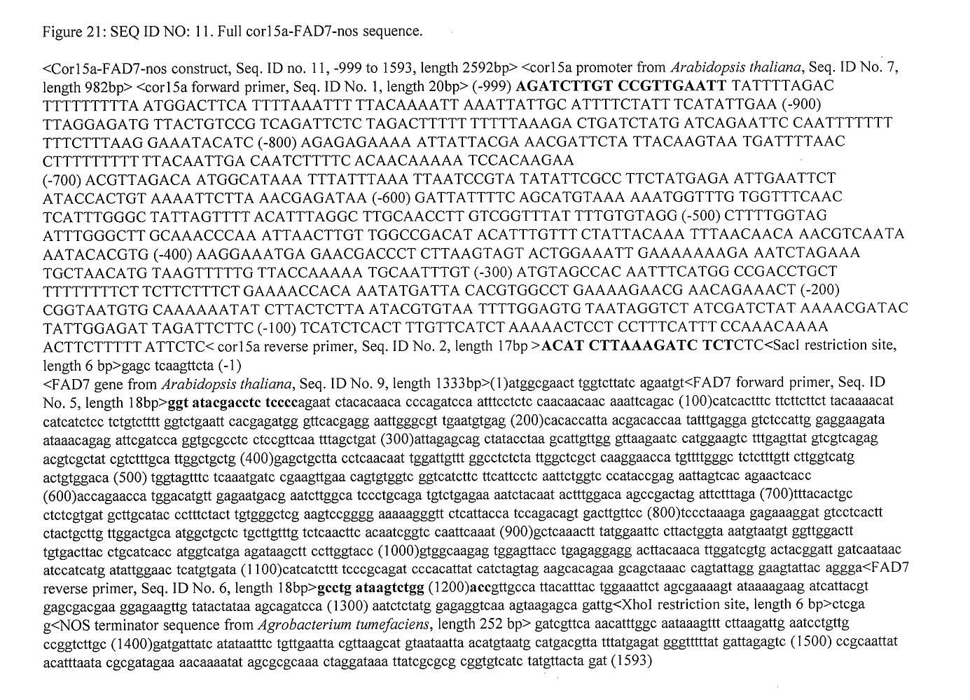

Quantitative analysis revealed that leaf chlorophyll concentrations in cor15a-ipt petunia lines and wild type plants were similar under normal glasshouse growing conditions and showed a similar decline under prolonged dark storage conditions (FIG. 4). However, when plants were first exposed to a cold-induction treatment, the chlorophyll concentration in the cor15a-ipt lines remained at the level of normal grown plants even when exposed to prolonged dark storage. Cold induction had no beneficial effect on chlorophyll stability in the wild type plants and chlorophyll concentrations showed a precipitous decline in response to dark storage. Experiments with excised leaves of wild type and cor-15a-ipt chrysanthemum produced a similar response (data not shown).

### EXAMPLE

### Changes in Endogenous Concentrations of Cytokinins in cor15a-ipt Plants

Analysis of endogenous cytokinins, in freeze dried petunia shoot tips from cor15a-ipt plants, revealed a dramatic increase after a cold-induction treatment compared to concentrations from wild type plants (Table 1). Expression of the cor15a-ipt gene in petunia especially affected zeatin and dihydrozeatin type cytokinins. In cor15a-ipt plants, exposure to a cold-induction period (3.5 days at 4° C.) resulted in the increase of the physiologically active cytokinin trans-zeatin and its riboside (>4-fold and >18-fold, respectively), as well as of the storage cytokinins zeatin nucleotide (>10-fold increase), zeatin O-glucoside (5-fold increase) and the cytokinin deactivation products zeatin 7-glucoside (>10-fold increase) and zeatin-9-glucoside (>7-fold increase). The dihydrozeatin type cytokinins followed the same trend, but the increase was less dramatic. From the isopentenyladenine type cytokinins only the level of the active base (isopentenyladenine) was slightly increased during ipt expression at 4° C. The concentration of isopentenyladenosine was considerably elevated under growth permissive conditions (25° C.) in both wild type and transformed plants.

In cor15a-ipt chrysanthemum plants, cold-induced ipt gene expression produced marked increases in both the storage cytokinin pool (P≦0.05) and the pool of physiologically active cytokinins (P≦0.05), but the total cytokinin pool (active, deactivated and storage foams combined) was not substantially altered (Table 2). In more detail, trans-zeatin concentrations were similar in wild type chrysanthemum, under both non-inducing (25° C.) and cold-inducing (4° C.) temperatures, and in non-induced cor15a-ipt plants (averaging 8.2 pmol/g DW). However, concentrations increased (P≦0.05) in cor15a-ipt plants exposed to a prolonged period (14 d) at 4° C. and those exposed to a short cold induction period (3.5 d) followed by the transfer to the 25° C. growth permissive conditions for 3.5 d (averaging 16.8 pmol/g DW). But, 10.5 d after transfer to 25° C. the concentration of trans-zeatin in cold induced cor15a-ipt plants decreased to non-induced concentrations. In cor15a-ipt plants exposed to 4° C. for either 7 d or for 3.5 d followed by 3.5 d at 25° C., the concentration of trans-zeatin riboside (averaging 6.6 pmol/g DW) was measurably higher (P≦0.05) than in non-induced cor15a-ipt plants and wild type plants in both inductive and non-inductive conditions (averaging 1.5 pmol/g DW). The concentration of isopentenyladenine detected in non-induced wild type, non-induced cor15a-ipt, and cold induced wild type plants (4.1 pmol/g DW), was higher (P≦0.05) than the concentration found in cor15a-ipt plants exposed to cold for between 3.5 d and 14 d, or in plants exposed to cold for 3.5 d and then returned to 25° C. for 3.5 d (2.7 pmol/g DW). In contrast, the concentration of the corresponding riboside (iP7R) significantly increased (P≦0.05) in cor15a-ipt plants induced in cold for 3.5 d and then returned to growth conditions for either 3.5 d or 10.5 d (13.1 pmol/g DW) compared to the average concentration found in cold-induced wild type plants, non-induced wild type plants and non-induced cor15a-ipt plants (7.1 pmol/g DW). The concentration of iP7R was dramatically lower (P≦0.01) in cor15a-ipt plants after 3.5 d of cold-induction than when similar plants were transferred to growth conditions for 3.5 d or 10.5 d. The concentration of dihydrozeatin was low in all plants held at 4° C., but the concentration of dihydrozeatin riboside increased with ipt expression.

### EXAMPLE

### Plant Morphology in Plants Harboring cor15a-ipt

The overall growth habit of cor15a-ipt plants under growth chamber conditions (25° C.) was not substantially different from the wild type chrysanthemum line (Table 3). In addition, the overall growth response of both cor15a-ipt lines and wild type plants that were first exposed to a cold-induction treatment remained similar, indicating that the increase in ipt expression in cold-induced plants did not have a long lasting effect on subsequent plant growth. Of the growth parameters observed only shoot fresh weight and average lateral shoot length were affected by genotype. Shoot fresh weight for cor15a-ipt line #12 was similar to the wild type while shoot fresh weight for cor15a-ipt line #9 was lower. However, average lateral shoot length for cor15a-ipt line #12 was greater than either the wild type or cor15-ipt line #9. Shoot fresh weight and average leaf size (on the upper most lateral branch) were both affected by cold-induction treatment but both the cor15a-ipt lines and the wild type plants responded in the same way to this treatment. Most significantly there was no interactive effect of genotype and environmental treatment on any of the growth responses observed, indicating that any increase in cytokinin that resulted from a cold-induction period did not persist during plant development at normal glasshouse temperatures. Average number of lateral branches on each plant, number of secondary branches on each lateral shoot and average internode length on the top lateral branch were all unaffected by genotype or temperature treatment.

No differences were observed between non-induced wild type and cor15a-ipt petunia lines grown in the 25° C. growth chamber. For example, the average length of the main stem of non-induced wild type [21 cm (standard error 3.0)] and cor15a-ipt petunia plants [20.1 cm (se 0.6)] were similar. Likewise, the average number of lateral shoots on the main stem [5.8 (se 1.2) and 6.8 (se 0.4)], and the average internode length on the main stem [1.7 cm (se 0.3) and 1.5 cm (se 0.1)] were also similar for wild type and cor15a-ipt petunia plants, respectively. Even when exposed to an initial cold treatment, growth response was similar for the wild type and the cor15a-ipt transgenic petunia lines for four of the six parameters measured (length of the main stem, number of leaves on the main shoot, leaf area on the main shoot, and number of lateral branches on the main shoot). Compared to the wild type, shorter internodes were observed on two of the three transgenic lines tested and one transgenic line displayed a leaf area increase on the first lateral shoot. None of these anatomical features were consistent with the type of changes associated with constitutive ipt gene expression.

### EXAMPLE

### Cold Tolerance and Survival Rate of Wild Type and cor15a-FAD7 Transgenic Plants

Although short-term cold-induction treatment (4 days at 4° C.) caused no visible injury in either cor15a-FAD7 transgenic or wild type tobacco seedlings, long-term exposure to cold (0.5 to 3.5° C.) caused visible injury and dramatically reduced survival in wild type plants. Chill injury first appeared as chlorosis and the degree and extent of injury became progressively more pronounced as exposure to the cold continued. After extended exposure to cold, most leaves on the transgenic cor15a-FAD7 remained green and the plants appeared healthy, but in contrast, wild type plants exhibited leaf damage, whole plant loss of turgor, shoot collapse and death.

After 44 days exposure to 0.5° C., 2° C., or 3.5° C., tobacco seedlings carrying the cor15a-FAD7 construct were more resistant to injury than the wild type and enjoyed dramatically higher survival rates (P≦0.001). Survival rate for wild type plants averaged 8.3% (averaged over the entire temperature range), while average survival for individual transgenic lines ranged from 54 to 79% (FIG. 8.). Survival rate varied with storage temperature (P≦0.001), with the highest survival rate observed at 2° C. for the wild type and all cor15a-FAD7 lines except N1 (which survived best at 3.5° C.). At 2° C. the survival rate of the wild type plants averaged 10.2% while the survival rate of cor15a-FAD7 lines N2 and N3 both averaged 96%.

### EXAMPLE

### Fatty Acid Composition in Leaves of cor15a-fad7 Transgenic Plants

The most abundant fatty acids detected in the leaves of wild type N. tabacum when grown at ambient temperatures were 16:0, 16:3, 18:2, and 18:3 (Table 4). In wild type plants exposed to cold treatment, there was a decline in the trienoic species, 16:3 (P≦0.05) and 18:3 (P≦0.05) and an increase in the dienoic species, 18:2 (P<0.05) and the saturated species, 18:0 (P<0.001). The change observed in the other major fatty acid species, 16:0 was not significant (P=0.07). Before exposure to cold induction, the fatty acid profile of the wild type and the FAD7 transgenic plants were similar with the exception of 18:0 (P<0.05) which was 2.5-times higher in the FAD7 plants. However after cold induction 16:3 and 18:3 were both higher in the FAD7 transgenic plants than in wild type plants (P≦0.01 and P≦0.001, respectively), and 18:0, 18:1 and 18:2 were all lower in the FAD7 transgenic plants than in wild type plants (P≦0.01, P≦0.001 and P≦0.001, respectively). In wild type plants exposed to cold induction the 16:3 levels declined 79% (P<0.05) while remaining stable in the FAD7 line N2 plants. In contrast, the level of 18:3 in wild type plants declined 20.6% after exposure to cold but increased 18.5% in FAD7 plants after exposure to cold. Also, after cold induction 18:2 increased by 58% in wild type plants (P≦0.05) but declined to non-detectable levels in FAD7 plants.

### EXAMPLE

### Effects of Prolonged Exposure to 4° C. on Chloroplast Ultrastructure and Chlorophyll Concentration

Thylakoid structure and organization appeared similar in chloroplasts from both wild type and cor15a-FAD7 transgenic prior to exposure to prolonged cold stress conditions (FIG. 9-I). However in wild type plants, exposure to cold stress resulted in extensive changes in chloroplast ultrastructure (FIG. 9-II a, b). After prolonged and continuous exposure to cold temperatures, micrographs of chloroplast from wild type plants revealed swelling, loss of granal stacking, and membrane disorganization typically associated with chloroplast death (FIG. 9-II a, b). In contrast, micrographs from cor15a-FAD7 leaves revealed that chloroplasts retained normal thylakoid structure and organization even after 40 days at 4° C. (FIG. 9-I c, d; FIG. 9-II c, d).

### EXAMPLE

### Chlorophyll Retention in cor15a-fad7 Leaves

Changes in leaf chlorophyll concentration coincided with changes in chloroplast ultrastructure. Prior to exposure to cold, leaf chlorophyll concentrations were initially similar in both wild type and cor15a-FAD7 plants (919 and 916 ug/g fresh weight respectively). However after 40 days of exposure to cold (4° C.) leaf chlorophyll concentrations were dramatically lower (P≦0.001) in surviving wild type plants (144 ug/g FW) than in the cor15a-FAD7 plants (677 ug/g FW). Compared to the concentrations observed prior to cold, chlorophyll concentrations declined by 80% in wild type plants but only declined 28% in cor15a-FAD7 plants exposed to the same conditions.

### EXAMPLE

### Enhanced Tolerance to Both Cold and Dark Conditions

Double transgenic Nicotiana alata seedlings were generated, by crossing cor15a-FAD7 and cor15a-IPT parent lines, and the tolerance of the double transgenic seedlings to exposure to both dark and cold conditions was evaluated. RT-PCR analysis confirmed cold-induced expression of both the IPT and FAD7 genes putative double transgenic seedlings (FIG. 10). No IPT expression was observed in either line #33 or line #41 in the absence of a cold-induction signal. No FAD7 expression was observed in line #33 in the absence of a cold-induction signal, but a low level of expression was observed in line #41.

Both double transgenic T₁ generation lines #33 and #41 resisted injury under prolonged cold, dark conditions. On average, survival of double transgenic Nicotiana alata plants was dramatically higher (90% for line #33 and 89% for line #41) than for wild type plants (2%) following prolonged exposure to cold, dark conditions (P≦0.001).

### EXAMPLE

### Fatty Acid Composition in Double Transgenic (cor15a-ipt×cor15a-fad7) N. alata Lines

Fatty acid analysis of Nicotiana alata leaves revealed that 16:0, 16:3, 18:2, and 18:3 were the major fatty acid species detected (Table 5) in wild type plants, while 16:0, 16:3 and 18:3 were the major fatty acid species detected in the double transgenic lines. The level of 18:3 was higher in the double transgenic (lines 41 and 33) plants than in wild type regardless of cold induction treatment (P≦0.01 under non-inductive conditions and P≦0.001 under cold inductive conditions). In response to cold-induction temperatures, 18:3 in wild type plants showed a marginal decline (P=0.08) and the 16:0 content showed a marginal increase (P=0.08). In the double transgenic lines, the 18:3 content remained stable after exposure to cold inductive temperatures and 16:3 increased 60% in line #41 (P≦0.05) and 49% in line #33 (P≦0.01), and the level of 16:0 decreased in both lines (P≦0.05). The fatty acid species 18:2 was detected only in wild type plants and was unaffected by cold inductive conditions.

What is claimed is:

1. A transgenic plant comprising
a first transgene capable of producing endogenous cytokinin, and
a second transgene capable of desaturating membrane fatty acids,
wherein each transgene is operably linked to a plant cold-regulated gene promoter.

2. The transgenic plant of claim 1, wherein the first transgene codes for an isopentyltransferase.

3. The transgenic plant of claim 1, wherein the second transgene codes for a fatty acid desaturase.

4. The transgenic plant of claim 1, wherein at least one promoter has the nucleotide sequence substantially identical to that set forth in SEQ ID NO: 7.

5. The transgenic plant of claim 1, wherein the first transgene has a nucleotide sequence substantially identical to the sequence in SEQ ID NO: 8.

6. The transgenic plant of claim 1, wherein the first transgene has the nucleotide sequence set forth in SEQ ID NO: 8.

7. The transgenic plant of claim 1, wherein the second transgene has a nucleotide sequence substantially identical to the sequence in SEQ ID NO: 9.

8. The transgenic plant of claim 1, wherein the second transgene has the nucleotide sequence set forth in SEQ ID NO: 9.

9. The transgenic plant of claim 1, wherein
the first transgene has the nucleotide sequence set forth in SEQ ID NO: 8,
the second transgene has the nucleotide sequence set forth in SEQ ID NO: 9, and
at least one promoter has the nucleotide sequence set forth in SEQ ID NO: 7.

10. The transgenic plant of claim 1, wherein the first transgene and its respective promoter have the nucleotide sequence set forth in SEQ ID NO: 10 and the second transgene and its respective promoter have the nucleotide sequence set forth in SEQ ID NO: 11.

11. A seed of a transgenic plant comprising
a first transgene capable of producing endogenous cytokinin, and
a second transgene capable of desaturating membrane fatty acids,
wherein each transgene is operably linked to a plant cold-regulated gene promoter.

12. The seed of claim 11, wherein the first transgene codes for an isopentyltransferase.

13. The seed of claim 11, wherein the second transgene codes for a fatty acid desaturase.

14. The seed of claim 11, wherein at least one promoter has the nucleotide sequence set forth in SEQ ID NO: 7.

15. The seed of claim 11, wherein the first transgene has the nucleotide sequence substantially identical to the sequence in SEQ ID NO: 8.

16. The seed of claim 11, wherein the first transgene has the nucleotide sequence set forth in SEQ ID NO: 8.

17. The seed of claim 11, wherein the second transgene has the nucleotide sequence substantially identical to the sequence in SEQ ID NO: 9.

18. The seed of claim 11, wherein the second transgene has the nucleotide sequence set forth in SEQ ID NO: 9.

19. The seed of claim 11, wherein
the first transgene has the nucleotide sequence set forth in SEQ ID NO: 8,
the second transgene has the nucleotide sequence set forth in SEQ ID NO: 9, and
at least one promoter has the nucleotide sequence set forth in SEQ ID NO: 7.

20. The seed of claim 11, wherein the first transgene and its respective promoter have the nucleotide sequence set forth in SEQ ID NO: 10 and the second transgene and its respective promoter have the nucleotide sequence set forth in SEQ ID NO: 11.


SEQUENCE LISTING (11 sequences)

SEQ ID NO: 1 - DNA, 20 bp, Artificial Sequence
  Primer used for DNA amplification of the 0.982kb fragment of the cor15a gene promoter. Forward primer 5′-AGATCTTGTCCGTTGAATTT-3′.
agatcttgtccgttgaattt

SEQ ID NO: 2 - DNA, 17 bp, Artificial Sequence
  Primer used for DNA amplification of the 0.982kb fragment of the cor15a gene promoter. Reverse primer 5′-AGAGATCTTTAAGATGT-3′.
agagatctttaagatgt

SEQ ID NO: 3 - DNA, 21 bp, Artificial Sequence
  Primer used for DNA amplification of the 0.523 kb region of ipt gene. Forward primer 5′-GGTCCAACTTGCACAGGAAAG-3′.
ggtccaacttgcacaggaaag

SEQ ID NO: 4 - DNA, 21 bp, Artificial Sequence
  Primer used for DNA amplification of the 0.523 kb region of ipt gene. Reverse primer 5′-TAACAAACAACATGGCATATC-3′.
taacaaacaacatggcatatc

SEQ ID NO: 5 - DNA, 18 bp, Artificial Sequence
  The primer used for DNA amplification of the 1.176 kb fragment of the FAD7 gene. Forward primer 5′-GGTATACGACCTCTCCCC-3′.
ggtatacgacctctcccc

SEQ ID NO: 6 - DNA, 18 bp, Artificial Sequence
  The primer used for DNA amplification of the 1.176 kb fragment of the FAD7 gene. Reverse primer 5′-GGTCCAGACTTATCAGGC-3′.
ggtccagacttatcaggc

SEQ ID NO: 7 - DNA, 982 bp, Arabidopsis thaliana
agatcttgtccgttgaatttattttagactttttttttaatggacttcattttaaatttttacaaaattaaattattgcattttctatttcatattgaattaggagatgttactgtccgtcagattctctagacttttttttttaaagactgatctatgatcagaattccaattttttttttctttaaggaaatacatcagagagaaaaattattacgaaacgattctattacaagtaatgattttaacctttttttttttacaattgacaatcttttcacaacaaaaatccacaagaaacgttagacaatggcataaatttatttaaattaatccgtatatattcgccttctatgagaattgaattctataccactgtaaaattcttaaacgagataagattattttcagcatgtaaaaaatggtttgtggtttcaactcatttgggctattagttttacatttaggcttgcaaccttgtcggtttattttgtgtaggcttttggtagatttgggcttgcaaacccaaattaacttgttggccgacatacatttgtttctattacaaatttaacaacaaacgtcaataaatacacgtgaaggaaatgagaacgaccctcttaagtagtactggaaattgaaaaaaagaaatctagaaatgctaacatgtaagtttttgttaccaaaaatgcaatttgtatgtagccacaatttcatggccgacctgctttttttttcttcttctttctgaaaaccacaaatatgattacacgtggcctgaaaagaacgaacagaaactcggtaatgtgcaaaaaatatcttactcttaatacgtgtaattttggagtgtaataggtctatcgatctataaaacgatactattggagattagattcttctcatctcactttgttcatctaaaaactcctcctttcatttccaaacaaaaacttctttttattctcacatcttaaagatctct

SEQ ID NO: 8 - DNA, 723 bp, Agrobacterium tumefaciens
atggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcacacgaggagaccttcatgaacgtggccaaggccagagttaagcagatgttacgccccgcttcaggcctttctattatccaagagttggttgatctttggaaagagcctcggctgaggcgcatactgaaagagatcgatggatatcgatatgccatgttgtttgttagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtattag

SEQ ID NO: 9 - DNA, 1333 bp, Arabidopsis thaliana
atggcgaacttggtcttatcagaatgtggtatacgacctctccccagaatctacacaacacccagatccaatttcctctccaacaacaacaaattcagaccatcactttcttcttcttcttacaaaacatcatcatctcctctgtcttttggtctgaattcacgagatgggttcacgaggaattgggcgttgaatgtgagcacaccattaacgacaccaatatttgaggagtctccattggaggaagataataaacagagattcgatccaggtgcgcctcctccgttcaatttagctgatattagagcagctatacctaagcattgttgggttaagaatccatggaagtctttgagttatgtcgtcagagacgtcgctatcgtctttgcattggctgctggagctgcttacctcaacaattggattgtttggcctctctattggctcgctcaaggaaccatgttttgggctctctttgttcttggtcatgactgtggacatggtagtttctcaaatgatccgaagttgaacagtgtggtcggtcatcttcttcattcctcaattctggtcccataccgagaattagtcacagaactcaccaccagaaccatggacatgttgagaatgacgaatcttggcatccctgcagatgtctgagaaaatctacaatactttggacaagccgactagattctttagatttacactgcctctcgtgatgcttgcataccctttctacttgtgggctcgaagtccggggaaaaagggttctcattaccatccagacagtgacttgttcctccctaaagagagaaaggatgtcctcacttctactgcttgttggactgcaatggctgctctgcttgtttgtctcaacttcacaatcggtccaattcaaatgctcaaactttatggaattccttactggtaaatgtaatgtggttggactttgtgacttacctgcatcaccatggtcatgaagataagcttccttggtaccgtggcaagagtggagttacctgagaggaggacttacaacattggatcgtgactacggattgatcaataacatccatcatgatattggaactcatgtgatacatcatcttttcccgcagatcccacattatcatctagtagaagcacagaagcagctaaaccagtattagggaagtattacagggagcctgataagtctggaccgttgccattacatttactggaaattctagcgaaaagtataaaagaagatcattacgtgagcgacgaaggagaagttgtatactataaagcagatccaaatctctatggagaggtcaaagtaagagcagat

SEQ ID NO: 10 - DNA, 1968 bp, Artificial Sequence
  Cor15a-ipt-nos
agatcttgtccgttgaatttattttagactttttttttaatggacttcattttaaatttttacaaaattaaattattgcattttctatttcatattgaattaggagatgttactgtccgtcagattctctagacttttttttttaaagactgatctatgatcagaattccaattttttttttctttaaggaaatacatcagagagaaaaattattacgaaacgattctattacaagtaatgattttaacctttttttttttacaattgacaatcttttcacaacaaaaatccacaagaaacgttagacaatggcataaatttatttaaattaatccgtatatattcgccttctatgagaattgaattctataccactgtaaaattcttaaacgagataagattattttcagcatgtaaaaaatggtttgtggtttcaactcatttgggctattagttttacatttaggcttgcaaccttgtcggtttattttgtgtaggcttttggtagatttgggcttgcaaacccaaattaacttgttggccgacatacatttgtttctattacaaatttaacaacaaacgtcaataaatacacgtgaaggaaatgagaacgaccctcttaagtagtactggaaattgaaaaaaagaaatctagaaatgctaacatgtaagtttttgttaccaaaaatgcaatttgtatgtagccacaatttcatggccgacctgctttttttttcttcttctttctgaaaaccacaaatatgattacacgtggcctgaaaagaacgaacagaaactcggtaatgtgcaaaaaatatcttactcttaatacgtgtaattttggagtgtaataggtctatcgatctataaaacgatactattggagattagattcttctcatctcactttgttcatctaaaaactcctcctttcatttccaaacaaaaacttctttttattctcacatcttaaagatctctctcatgtcgagatggatctgcgtctaattttcggtccaacttgcacaggaaagacgtcgaccgcggtagctcttgcccagcagactgggcttccagtcctttcgctcgatcgggtccaatgttgtcctcagctgtcaaccggaagcggacgaccaacagtggaagaactgaaaggaacgagccgtctataccttgatgatcggcctctggtgaagggtatcatcgcagccaagcaagctcatgaaaggctgatgggggaggtgtataattatgaggcccacggcgggcttattcttgagggaggatctatctcgttgctcaagtgcatggcgcaaagcagttattggagtgcggattttcgttggcatattattcgccacgagttagcacacgaggagaccttcatgaacgtggccaaggccagagttaagcagatgttacgccccgcttcaggcctttctattatccaagagttggttgatctttggaaagagcctcggctgaggcgcatactgaaagagatcgatggatatcgatatgccatgttgtttgttagccagaaccagatcacatccgatatgctattgcagcttgacgcagatatggaggataagttgattcatgggatcgctcaggagtatctcatccatgcacgccgacaagaacagaaattccctcgagttaacgcagccgcttacgacggattcgaaggtcatccattcggaatgtattaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat

SEQ ID NO: 11 - DNA, 2595 bp, Artificial Sequence
  Cor15a-fad7-nos
agatcttgtccgttgaatttattttagactttttttttaatggacttcattttaaatttttacaaaattaaattattgcattttctatttcatattgaattaggagatgttactgtccgtcagattctctagacttttttttttaaagactgatctatgatcagaattccaattttttttttctttaaggaaatacatcagagagaaaaattattacgaaacgattctattacaagtaatgattttaacctttttttttttacaattgacaatcttttcacaacaaaaatccacaagaaacgttagacaatggcataaatttatttaaattaatccgtatatattcgccttctatgagaattgaattctataccactgtaaaattcttaaacgagataagattattttcagcatgtaaaaaatggtttgtggtttcaactcatttgggctattagttttacatttaggcttgcaaccttgtcggtttattttgtgtaggcttttggtagatttgggcttgcaaacccaaattaacttgttggccgacatacatttgtttctattacaaatttaacaacaaacgtcaataaatacacgtgaaggaaatgagaacgaccctcttaagtagtactggaaattgaaaaaaagaaatctagaaatgctaacatgtaagtttttgttaccaaaaatgcaatttgtatgtagccacaatttcatggccgacctgctttttttttcttcttctttctgaaaaccacaaatatgattacacgtggcctgaaaagaacgaacagaaactcggtaatgtgcaaaaaatatcttactcttaatacgtgtaattttggagtgtaataggtctatcgatctataaaacgatactattggagattagattcttctcatctcactttgttcatctaaaaactcctcctttcatttccaaacaaaaacttctttttattctcacatcttaaagatctctctcatggagctcaagttctaatggcgaacttggtcttatcagaatgtggtatacgacctctccccagaatctacacaacacccagatccaatttcctctccaacaacaacaaattcagaccatcactttcttcttcttcttacaaaacatcatcatctcctctgtcttttggtctgaattcacgagatgggttcacgaggaattgggcgttgaatgtgagcacaccattaacgacaccaatatttgaggagtctccattggaggaagataataaacagagattcgatccaggtgcgcctcctccgttcaatttagctgatattagagcagctatacctaagcattgttgggttaagaatccatggaagtctttgagttatgtcgtcagagacgtcgctatcgtctttgcattggctgctggagctgcttacctcaacaattggattgtttggcctctctattggctcgctcaaggaaccatgttttgggctctctttgttcttggtcatgactgtggacatggtagtttctcaaatgatccgaagttgaacagtgtggtcggtcatcttcttcattcctcaattctggtcccataccgagaattagtcacagaactcaccaccagaaccatggacatgttgagaatgacgaatcttggcatccctgcagatgtctgagaaaatctacaatactttggacaagccgactagattctttagatttacactgcctctcgtgatgcttgcataccctttctacttgtgggctcgaagtccggggaaaaagggttctcattaccatccagacagtgacttgttcctccctaaagagagaaaggatgtcctcacttctactgcttgttggactgcaatggctgctctgcttgtttgtctcaacttcacaatcggtccaattcaaatgctcaaactttatggaattccttactggtaaatgtaatgtggttggactttgtgacttacctgcatcaccatggtcatgaagataagcttccttggtaccgtggcaagagtggagttacctgagaggaggacttacaacattggatcgtgactacggattgatcaataacatccatcatgatattggaactcatgtgatacatcatcttttcccgcagatcccacattatcatctagtagaagcacagaagcagctaaaccagtattagggaagtattacagggagcctgataagtctggaccgttgccattacatttactggaaattctagcgaaaagtataaaagaagatcattacgtgagcgacgaaggagaagttgtatactataaagcagatccaaatctctatggagaggtcaaagtaagagcagattgctcgaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagat